SEQUENCE LISTING (32 sequences)

SEQ ID NO: 1 - protein, 127 aa, Homo sapiens
QVQLVESGGTLVQPGGSLRLSCAASGFTFSSYAMSWIRQAPGKGLEWVSGISASGGYIDYADSVKGRVTISRDNSKNMAYLQMSSLRAEDTALYYCAKGGNYQMLLDHWGQGTLVTVSSASTKGPKL

SEQ ID NO: 2 - protein, 125 aa, Homo sapiens
QVQLVQSGAEVKKDGASVKVSCKATGGTFSGHAISWVRQAPGQRLEWMGEISPMFGTPNYAQSFQGRVTITADESTSYMEVSSLRSEDTATYYCARGANYRALLDYWGQGTLVTVSSASTKGPKL

SEQ ID NO: 3 - protein, 134 aa, Homo sapiens
QVQLVESGGGLVQPGGSLRLSCAASGFTFSNYWMSWVRQAPGKGLEWVANIKQDGSEKYYVDSVKGRFTISRDNAKNSLYLQMNSLRAEDTAVYYCARGSLCTDGSCPTIGPGPNWGQGTLVTVSSAPTKAPKL

SEQ ID NO: 4 - protein, 118 aa, Homo sapiens
DIQMTQSPSSLSASTGDRVTITCRASQDISSYLAWYQQAPGKAPHLLMSGATTLQTGVPSRFSGSGSGTDFTLTITSLQSEDFATYYCQQYYIYPPTFGQGTRVEIKRTVAAPSVFAA

SEQ ID NO: 5 - DNA, 381 bp, Homo sapiens
caggtacagctggtggagtctgggggaaccttggtacagcctggggggtccctgagactctcctgtgcagcctctggattcacctttagcagctatgccatgagctggatccgccaggctccagggaaggggctggagtgggtctcaggtattagtgctagtggtggttatatagactatgccgattccgtgaagggccgggtcaccatctccagagacaattccaagaacatggcatatctacaaatgagcagcctgagagccgaggacacggccctttattactgtgcgaaaggaggcaactaccagatgctattggaccactggggccagggaaccctggtcaccgtctcctcagcctccaccaagggcccaaagctt

SEQ ID NO: 6 - DNA, 375 bp, Homo sapiens
caggtacagctggtgcagtctggggctgaagtgaagaaggatggggcctcagtgaaggtctcctgcaaggctactggaggcactttcagcggtcacgctatcagttgggtgcgacaggcccctgggcaaagacttgagtggatgggggagatcagccctatgtttggaacaccaaactacgcacagagcttccagggcagagtcacgattaccgcggacgaatctacgagttacatggaggtgagcagcctgagatctgaggacacggccacttattactgtgcgagaggtgcgaactaccgggccctccttgattactggggccagggaaccctggtcaccgtctcctcagcctccaccaagggcccaaagctt

SEQ ID NO: 7 - DNA, 402 bp, Homo sapiens
caggtacagctggtggagtctgggggaggcttggtccagcctggggggtccctgagactctcctgtgcagcctctggattcacctttagtaactattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagaggttcactctgtactgatggtagctgccccaccatagggcctgggccaaactggggccagggaaccctggtcaccgtctcctcagcacccaccaaggctccgaagctt

SEQ ID NO: 8 - DNA, 356 bp, Homo sapiens
gacatccagatgacccagtctccatcctcactctctgcatctacaggagacagagtcaccatcacttgtcgggcgagtcaagatattagcagttatttagcctggtatcaacaggcacccgggaaagcccctcatctcctgatgtctggagcaaccactttacagactggagtcccatcaaggttcagcggcagtggatctgggacagatttcactctcaccatcacgtccctgcagtctgaagattttgcaacttattactgtcaacagtattatatttaccctccgacgttcggccaagggaccagggtggaaatcaaacgaactgtggctgcaccatctgtcttcgcggccgc

SEQ ID NO: 9 - protein, 132 aa, Artificial Sequence
  Description of Artificial Sequence Humanised Antibody
QVQLQQSGAEVKKPGSSVKVSCKASGGTFSTHTINWVRQAPGQGLEWMGGIAPMFGTANYAQKFQGRVTITADKSTSTAYMEMSSLRSDDTAVYYCARRRIAYGYDEGHAMDYWGQGTLVTVSSASTKGPKL

SEQ ID NO: 10 - protein, 132 aa, Artificial Sequence
  Description of Artificial Sequence Humanised Antibody
QVQLVQSGAELKKPGSSMKVSCKASGDTFSTYSINWVRQAPGQGLEWMGVINPSGGSTSYAQKFQGRVTMTRDTSTSTVYMELSSLRSEDTAVYYCARRRIAYGYDEGHAMDYWGQGTLVTVSSASTKGPKL

SEQ ID NO: 11 - protein, 118 aa, Artificial Sequence
  Description of Artificial Sequence Humanised Antibody
DIQMTQSPSSLSASTGDRVTITCRASQDISSYLAWYQQAPGKAPHLLMSGATTLQTGVPSRFSGSGSGTDFTLTISSLQAEDVAVYYCQQYYRTPFTFGQGTKLEIKRTVAAPSVFAA

SEQ ID NO: 12 - DNA, 396 bp, Artificial Sequence
  Description of Artificial Sequence Humanised Antibody
caggtacagctgcagcagtcaggggctgaggtgaagaagcctgggtcctcggtgaaggtctcctgcaaggcttctggaggcaccttcagcacccatactatcaactgggtgcgacaggcccctggacaagggcttgagtggatgggagggatcgcccctatgtttggtacagcaaactacgcacagaagttccagggcagagtcacaattaccgcggacaaatccacgagcacagcctacatggagatgagcagcctgagatctgacgacacggctgtgtattactgtgcaagaagaagaatcgcgtacggttacgacgagggccatgctatggactactggggtcaaggaacccttgtcaccgtctcctcagcctccaccaaggggccaaagctt

SEQ ID NO: 13 - DNA, 396 bp, Knstliche Sequenz
  Description of Artificial Sequence Humanised Antibody
caggtgcagctggtgcagtctggggctgagttgaagaagcctgggtcctcgatgaaggtctcctgcaaggcttctggagacaccttcagcacctattctatcaactgggtgcgacaggcccctggacaagggcttgagtggatgggagtaatcaaccctagtggtggtagcacaagctacgcacagaagttccagggcagagtcaccatgaccagggacacgtccacgagcacagtttacatggagctgagcagcctgagatctgaagacacggccgtgtattactgtgcgagaagaagaatcgcgtacggttacgacgagggccatgctatggactactggggtcaaggaacccttgtcaccgtctcctcagcctccaccaagggcccaaagctt

SEQ ID NO: 14 - DNA, 356 bp, Artificial Sequence
  Description of Artificial Sequence Humanised Antibody
gacatccagatgacccagtctccatcctcactctctgcatctacaggagacagagtcaccatcacttgtcgggcgagtcaagatattagcagttatttagcctggtatcaacaggcacccgggaaagcccctcatctcctgatgtctggagcaaccactttacagactggagtcccatcaaggttcagcggcagtggatctgggacagatttcactctcaccatcagcagcctgcaggctgaagatgtggcagtttattactgtcagcaatattatcgtactccgtttacttttggccaggggaccaagttggagatcaaacgaactgtggctgcaccatctgtcttcgcggccgc

SEQ ID NO: 15 - protein, 255 aa, Homo sapiens
QVQLVESGGTLVQPGGSLRLSCAASGFTFSSYAMSWIRQAPGKGLEWVSGISASGGYIDYADSVKGRVTISRDNSKNMAYLQMSSLRAEDTALYYCAKGGNYQMLLDHWGQGTLVTVSSASTKGPKLEEGEFSEARVDIQMTQSPSSLSASTGDRVTITCRASQDISSYLAWYQQAPGKAPHLLMSGATTLQTGVPSRFSGSGSGTDFTLTITSLQSEDFATYYCQQYYIYPPTFGQGTRVEIKRTVAAPSVFAA

SEQ ID NO: 16 - protein, 260 aa, Artificial Sequence
  Description of Artificial Sequence Humanised Antibody
QVQLVQSGAELKKPGSSMKVSCKASGDTFSTYSINWVRQAPGQGLEWMGVINPSGGSTSYAQKFQGRVTMTRDTSTSTVYMELSSLRSEDTAVYYCARRRIAYGYDEGHAMDYWGQGTLVTVSSASTKGPKLEEGEFSEARVDIQMTQSPSSLSASTGDRVTITCRASQDISSYLAWYQQAPGKAPHLLMSGATTLQTGVPSRFSGSGSGTDFTLTISSLQAEDVAVYYCQQYYRTPFTFGQGTKLEIKRTVAAPSVFAA

SEQ ID NO: 17 - protein, 260 aa, Artificial Sequence
  Description of Artificial Sequence Humanised Antibody
QVQLQQSGAEVKKPGSSVKVSCKASGGTFSTHTINWVRQAPGQGLEWMGGIAPMFGTANYAQKFQGRVTITADKSTSTAYMEMSSLRSDDTAVYYCARRRIAYGYDEGHAMDYWGQGTLVTVSSASTKGPKLEEGEFSEARVDIQMTQSPSSLSASTGDRVTITCRASQDISSYLAWYQQAPGKAPHLLMSGATTLQTGVPSRFSGSGSGTDFTLTISSLQAEDVAVYYCQQYYRTPFTFGQGTKLEIKRTVAAPSVFAA

SEQ ID NO: 18 - protein, 253 aa, Homo sapiens
QVQLVQSGAEVKKDGASVKVSCKATGGTFSGHAISWVRQAPGQRLEWMGEISPMFGTPNYAQSFQGRVTITADESTSYMEVSSLRSEDTATYYCARGANYRALLDYWGQGTLVTVSSASTKGPKLEEGEFSEARVDIQMTQSPSSLSASTGDRVTITCRASQDISSYLAWYQQAPGKAPHLLMSGATTLQTGVPSRFSGSGSGTDFTLTITSLQSEDFATYYCQQYYIYPPTFGQGTRVEIKRTVAAPSVFAA

SEQ ID NO: 19 - protein, 262 aa, Homo sapiens
QVQLVESGGGLVQPGGSLRLSCAASGFTFSNYWMSWVRQAPGKGLEWVANIKQDGSEKYYVDSVKGRFTISRDNAKNSLYLQMNSLRAEDTAVYYCARGSLCTDGSCPTIGPGPNWGQGTLVTVSSAPTKAPKLEEGEFSEARVDIQMTQSPSSLSASTGDRVTITCRASQDISSYLAWYQQAPGKAPHLLMSGATTLQTGVPSRFSGSGSGTDFTLTITSLQSEDFATYYCQQYYIYPPTFGQGTRVEIKRTVAAPSVFAA

SEQ ID NO: 20 - DNA, 767 bp, Homo sapiens
caggtacagctggtggagtctgggggaaccttggtacagcctggggggtccctgagactctcctgtgcagcctctggattcacctttagcagctatgccatgagctggatccgccaggctccagggaaggggctggagtgggtctcaggtattagtgctagtggtggttatatagactatgccgattccgtgaagggccgggtcaccatctccagagacaattccaagaacatggcatatctacaaatgagcagcctgagagccgaggacacggccctttattactgtgcgaaaggaggcaactaccagatgctattggaccactggggccagggaaccctggtcaccgtctcctcagcctccaccaagggcccaaagcttgaagaaggtgaattttcagaagcacgcgtagacatccagatgacccagtctccatcctcactctctgcatctacaggagacagagtcaccatcacttgtcgggcgagtcaagatattagcagttatttagcctggtatcaacaggcacccgggaaagcccctcatctcctgatgtctggagcaaccactttacagactggagtcccatcaaggttcagcggcagtggatctgggacagatttcactctcaccatcacgtccctgcagtctgaagattttgcaacttattactgtcaacagtattatatttaccctccgacgttcggccaagggaccagggtggaaatcaaacgaactgtggctgcaccatctgtcttcgcggccgc

SEQ ID NO: 21 - DNA, 782 bp, Artificial Sequence
  Description of Artificial Sequence Humanised Antibody
caggtgcagctggtgcagtctggggctgagttgaagaagcctgggtcctcgatgaaggtctcctgcaaggcttctggagacaccttcagcacctattctatcaactgggtgcgacaggcccctggacaagggcttgagtggatgggagtaatcaaccctagtggtggtagcacaagctacgcacagaagttccagggcagagtcaccatgaccagggacacgtccacgagcacagtttacatggagctgagcagcctgagatctgaagacacggccgtgtattactgtgcgagaagaagaatcgcgtacggttacgacgagggccatgctatggactactggggtcaaggaacccttgtcaccgtctcctcagcctccaccaagggcccaaagcttgaagaaggtgaattttcagaagcacgcgtagacatccagatgacccagtctccatcctcactctctgcatctacaggagacagagtcaccatcacttgtcgggcgagtcaagatattagcagttatttagcctggtatcaacaggcacccgggaaagcccctcatctcctgatgtctggagcaaccactttacagactggagtcccatcaaggttcagcggcagtggatctgggacagatttcactctcaccatcagcagcctgcaggctgaagatgtggcagtttattactgtcagcaatattatcgtactccgtttacttttggccaggggaccaagttggagatcaaacgaactgtggctgcaccatctgtcttcgcggccgc

SEQ ID NO: 22 - DNA, 782 bp, Artificial Sequence
  Description of Artificial Sequence Humanised Antibody
caggtacagctgcagcagtcaggggctgaggtgaagaagcctgggtcctcggtgaaggtctcctgcaaggcttctggaggcaccttcagcacccatactatcaactgggtgcgacaggcccctggacaagggcttgagtggatgggagggatcgcccctatgtttggtacagcaaactacgcacagaagttccagggcagagtcacaattaccgcggacaaatccacgagcacagcctacatggagatgagcagcctgagatctgacgacacggctgtgtattactgtgcaagaagaagaatcgcgtacggttacgacgagggccatgctatggactactggggtcaaggaacccttgtcaccgtctcctcagcctccaccaaggggccaaagcttgaagaaggtgaattttcagaagcacgcgtagacatccagatgacccagtctccatcctcactctctgcatctacaggagacagagtcaccatcacttgtcgggcgagtcaagatattagcagttatttagcctggtatcaacaggcacccgggaaagcccctcatctcctgatgtctggagcaaccactttacagactggagtcccatcaaggttcagcggcagtggatctgggacagatttcactctcaccatcagcagcctgcaggctgaagatgtggcagtttattactgtcagcaatattatcgtactccgtttacttttggccaggggaccaagttggagatcaaacgaactgtggctgcaccatctgtcttcgcggccgc

SEQ ID NO: 23 - DNA, 761 bp, Homo sapiens
caggtacagctggtgcagtctggggctgaagtgaagaaggatggggcctcagtgaaggtctcctgcaaggctactggaggcactttcagcggtcacgctatcagttgggtgcgacaggcccctgggcaaagacttgagtggatgggggagatcagccctatgtttggaacaccaaactacgcacagagcttccagggcagagtcacgattaccgcggacgaatctacgagttacatggaggtgagcagcctgagatctgaggacacggccacttattactgtgcgagaggtgcgaactaccgggccctccttgattactggggccagggaaccctggtcaccgtctcctcagcctccaccaagggcccaaagcttgaagaaggtgaattttcagaagcacgcgtagacatccagatgacccagtctccatcctcactctctgcatctacaggagacagagtcaccatcacttgtcgggcgagtcaagatattagcagttatttagcctggtatcaacaggcacccgggaaagcccctcatctcctgatgtctggagcaaccactttacagactggagtcccatcaaggttcagcggcagtggatctgggacagatttcactctcaccatcacgtccctgcagtctgaagattttgcaacttattactgtcaacagtattatatttaccctccgacgttcggccaagggaccagggtggaaatcaaacgaactgtggctgcaccatctgtcttcgcggccgc

SEQ ID NO: 24 - DNA, 788 bp, Homo sapiens
caggtacagctggtggagtctgggggaggcttggtccagcctggggggtccctgagactctcctgtgcagcctctggattcacctttagtaactattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagaggttcactctgtactgatggtagctgccccaccatagggcctgggccaaactggggccagggaaccctggtcaccgtctcctcagcacccaccaaggctccgaagcttgaagaaggtgaattttcagaagcacgcgtagacatccagatgacccagtctccatcctcactctctgcatctacaggagacagagtcaccatcacttgtcgggcgagtcaagatattagcagttatttagcctggtatcaacaggcacccgggaaagcccctcatctcctgatgtctggagcaaccactttacagactggagtcccatcaaggttcagcggcagtggatctgggacagatttcactctcaccatcacgtccctgcagtctgaagattttgcaacttattactgtcaacagtattatatttaccctccgacgttcggccaagggaccagggtggaaatcaaacgaactgtggctgcaccatctgtcttcgcggccgc

SEQ ID NO: 25 - DNA, 17 bp, Primer
tacaggatccacgcgta

SEQ ID NO: 26 - DNA, 18 bp, Primer
tgacaagcttgcggccgc

SEQ ID NO: 27 - DNA, 21 bp, Primer
tctcgcacagtaatacacggc

SEQ ID NO: 28 - DNA, 24 bp, Primer
tctgtgtgcacagtaatatctggc

SEQ ID NO: 29 - DNA, 21 bp, Primer
tctcgcacagtaatacatggc

SEQ ID NO: 30 - DNA, 21 bp, Primer
tcttgcacagtaatacacagc

SEQ ID NO: 31 - DNA, 15 bp, Primer
gaataggccatggcg

SEQ ID NO: 32 - DNA, 25 bp, Primer
gggggcgggcgtacgcgattcttct

(19) United States
(12) Patent Application Publication
(10) Pub. No.: US 2002/0099180 A1
(43) Pub. Date: Jul. 25, 2002

(54) Human FAP-alpha-specific antibodies

(76) Inventors: Klaus Pfizenmaier, Tiefenbronn (DE); Dieter Moosmayer, Berlin (DE); Michael Mersmann, Braunschweig (DE); Alexej Schmidt, Leonberg (DE); John-Edward Park, Biberach (DE); Pilar Garin-Chesa, Biberach (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/811,737

(22) Filed: Mar. 19, 2001

Related U.S. Application Data
(60) Provisional application No. 60/201,009, filed on May 1, 2000.

(30) Foreign Application Priority Data
Mar. 17, 2000 (DE) 100 13 286.3
Sep. 11, 2000 (GB) 00 22 216.6

(51) Int. Cl. C07K 16/40

(52) U.S. Cl. 530/388.24; 530/388.26

(57) ABSTRACT

The invention relates to antibody proteins which specifically bind fibroblast activating protein alpha (FAPα). The invention further relates to the use of said antibodies for diagnostic and therapeutic purposes as well as processes for preparing said antibodies.

### RELATED APPLICATION

The benefit of prior provisional application Ser. No. 60/201,009, filed May 1, 2000 is hereby claimed.

### FIELD OF THE INVENTION

The invention relates to antibody proteins which specifically bind fibroblast activating protein alpha (FAPα). The invention further relates to the use of said antibodies for diagnostic and therapeutic purposes as well as processes for preparing said antibodies.

### BACKGROUND OF THE INVENTION

Massive growth of epithelial cell cancer is associated with a number of characteristic cellular and molecular changes in the surrounding stroma cells. One highly consistent feature of the reactive stroma of numerous types of epithelial cell cancer is the induction of the fibroblast activating protein alpha (from now on referred to as FAPα or FAP), a cell surface molecule of the reactive stromal fibroblast which was originally identified with the monoclonal antibody F19 (Garin-Chesa P., Old L. J. and Rettig W. J.; 1990; Proc Natl. Acad. Sci. 87:7235). Since the FAP is selectively expressed in stroma of a number of epithelial cell carcinomas, irrespective of the site and histological type of the carcinoma, it was desirable to develop a treatment concept for the FAPα target molecule in order to allow imaging techniques, the diagnosis and treatment of epithelial cell cancer and many other syndromes. For this purpose a monoclonal murine antibody named F19 was developed which specifically binds to FAP. This antibody was described in U.S. Pat. No. 5,059,523 and WO 93/05804 which are included in their entirety in this document by reference. A serious problem arises when non-human antibodies are used for in vivo applications in humans, i.e. they rapidly elicit an immune response to the foreign antigen. In the worst case, such an immune response against the antibody used may trigger anaphylactic shock. This drastically reduces the efficiency of the antibody in the patient and has an adverse effect on further use or makes any further use impossible. The humanisation of non-human antibodies is usually achieved by one of two methods:

(1) By the construction of non-human/human chimeric antibodies in which the non-human variable regions are coupled to the human constant regions (Boulianne G. L., Hozumi N. and Shulman, M. J. (1984)) Nature 312:643) or

(2) By replacing the complementarity determining regions (CDRs) in human variable regions with those of the non-human variable region and then coupling the newly formed humanised variable regions to human constant regions (Riechmann L., Clark M., Waldmann H. and Winter G. (1988) Nature 332:323).

Chimeric antibodies consist of fewer foreign protein sequences than non-human antibodies and therefore have a lesser xenoantigenic potential. Nevertheless, chimeric antibodies of this kind may trigger an immune reaction on account of the non-human V-regions in humans (LoBuglio A. F., Wheeler R. H., Trang J., Haynes A., Roger K., Harvey E. B., Sun L., Ghrayeb J. and Khazaeli M. B. (1989) Proc.Natl.Acad.Sci.86:4220). CDR-transmitted or newly formed humanised antibodies admittedly contain fewer foreign protein sequences in the V-regions, but these humanised antibodies are still capable of triggering an immune response in humans. W099/57151 A2 describes FAPα− specific humanised antibodies of this kind in which the humanisation has been achieved by transferring all 6 CDR regions (3 from the light chain, 3 from the heavy) from the corresponding F19 murine antibody. These antibodies still contain parts of the murine framework region.

The problem of the present invention is to provide improved FAPα-specific antibodies which overcome the above disadvantages of the prior art.

### SUMMARY OF THE INVENTION

The invention relates to antibody proteins which specifically bind fibroblast activating protein alpha (FAPα). The invention further relates to the use of said antibodies for diagnostic and therapeutic purposes as well as processes for preparing said antibodies.

### DESCRIPTION OF THE FIGURES

FIG. 1: HCDR3-retaining guided selection

FIG. 2: Schematic representation of the HCDR3 sequence with the integrated SplI (Pf23II)

FIG. 3: Binding of scFv #13 (minibody format) to FAP+-cells (FACS analyses)

FIG. 4: Primers used for PCR amplification of the human V repertoire

FIG. 5: Primers for amplifying the human VH-gene segment repertoire for the HCDR3 retaining guided selection process

FIG. 6: Sequences of the selected human FAP-specific VL regions

FIG. 7: Ag specificity of selected chimeric scFv. ELISA wells were coated with FAP or irrelevant Ag. TTX: tetanus toxoid; BSA: bovine serum albumin; HSA: human serum albumin; TF: transferrin; CHY: chymotrypsinogen; LYS: lysozyme; Detection was done with 9E10 and POD-labeled goat anti-mouse serum. Data are derived from triplicate values.

FIG. 8: Epitope specificity of selected chimeric scFv. Different concentrations of competitor were mixed with the respective scFv and added to FAP coated ELISA wells. The applied competitors were: cF19 (chimeric F19, with murine variable and constant human regions); hu IgG (unspecific human IgG serum). Detection was done as in FIG. 1. Data are from double values.

FIG. 9: Construction of the human VH gene segment library with retained HCDR3 F19. Schematic drawing of the final construct of VH, linker, VL and phage protein gpIII. By creation of a new restriction site the VH segment repertoire could be ligated to the pre-existing HCDR3 F19, linked later to the selected human VLs.

FIG. 10: Ag specificity of selected humanized scFv. Coating of ELISA wells and detection was carried out as in FIG. 1. PLA: plastic

FIG. 11: Binding of humanized scFv and Mb to cell surface-bound FAP analysed by flow cytometry. A) Binding of scFv #18 and #34 to FAP⁺cells. Cells were incubated with 100-200 nM scFv from E. coli extracts. B) Binding of Mb #18 and #34 to FAP⁺cells. Supernatants of P. mirabilis LVI containg 20 nM MB. C: Control binding of scFv F19 (purified by IMAC) to FAP⁺cells. Area for binding to FAP⁻control cells is gray. scFv were detected by 9E10 and FITC-labled Fc-specific anti-mouse serum, Mb by FITC-labeled Fc-specific anti-human serum. Each curve represents cytometer values of 5,000 predefined and measured events.

FIG. 12: Epitope specificity of humanized scFv for cellbound FAP. Different concentrations of competitor were mixed with the respective scFv and added to FAP⁺cells. cF19: chimeric F19 (chimeric F19, with murine variable and constant human regions); hu IgG: unspecific human IgG serum. Detection by 9E10 and FITC-labeled Fc-specific anti-mouse serum. Data represent cytometer values of 10,000 predefined and measured events.

FIG. 13: Assessment of apparent affinity for Mb #34 on FAP⁺cells. Mb #34 was purified by IMAC and size exclusion chromatography. Data are derived from the cytometer with values of 10,000 events for each Ab concentration after detection with FITC-labeled Fc-specific anti-human serum.

FIG. 14: Long term stability of Mb #34 at 37° C. After incubation in a tenfold volume of RPMI (5% FCS) for 0 to 42 h, the IMAC purified Mb was diluted and used in an anti-FAP ELISA. Detection was carried out with POD-labeled anti-human serum. Data are based on triplicate values.

FIG. 15: Immunohistological staining of biopsy material from FAP⁺tumor sections with Mb #34. Cryo-sections of A) breast carcinoma B) colon carcinoma C) lung carcinoma D) desmoid tumor E) malignant fibrous histiocytoma were stained with Mb #34. Bound Mb was detected by subsequent treatment of the section with an anti-c-myc mAb (9E10), a biotinylated horse anti-mouse serum and the avidin-biotin immunoperoxidase complex. As a negative control F) a cryo-section was only treated with the detection antibodies and the avidin-biotin immunoperoxidase complex.

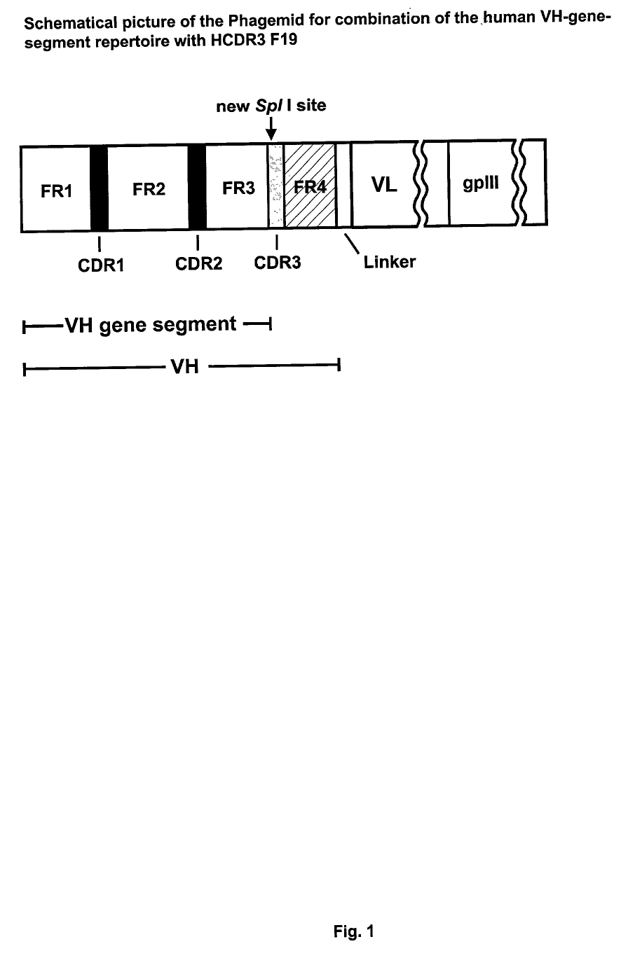

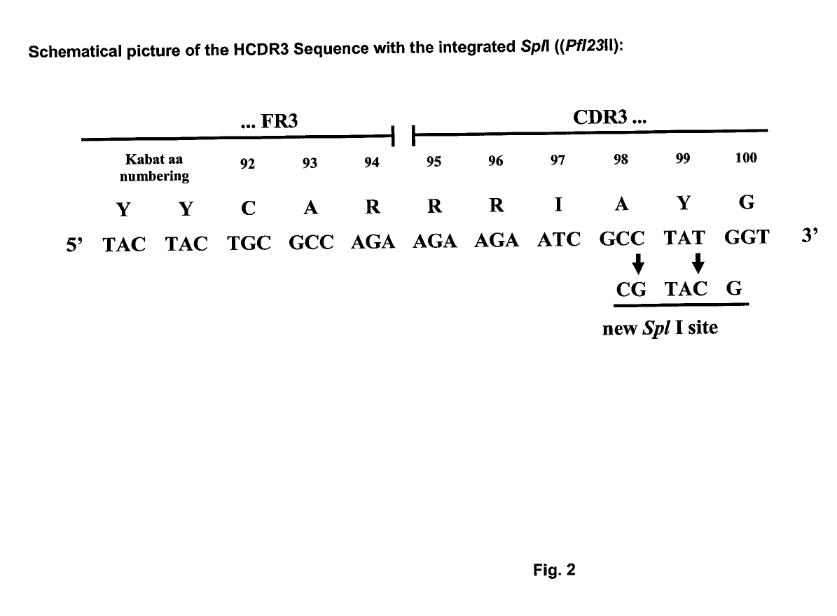

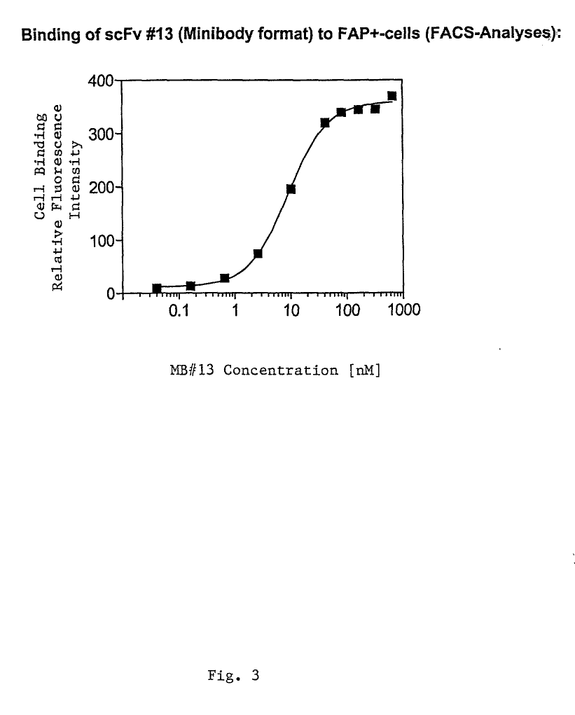

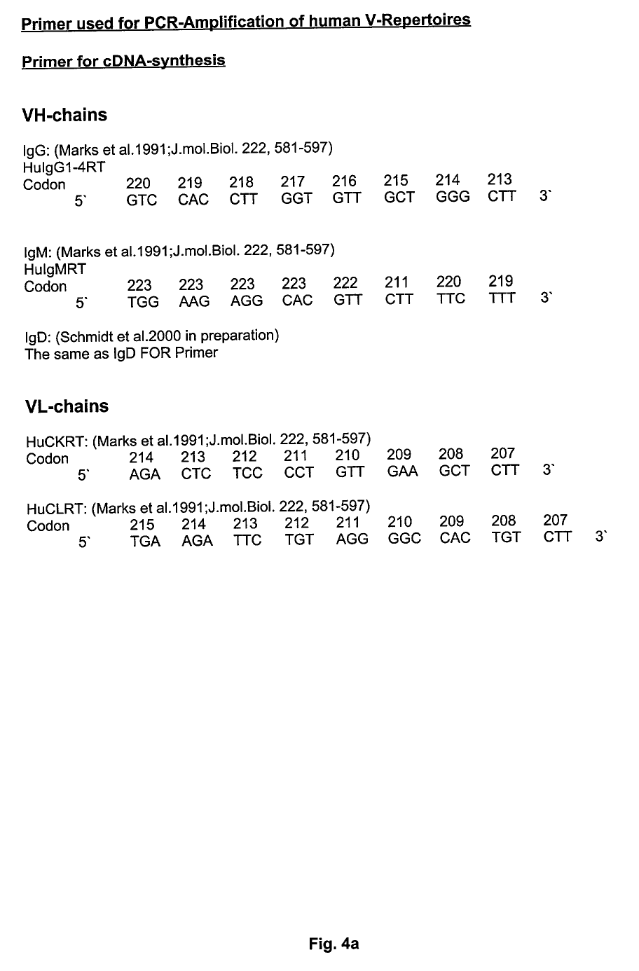

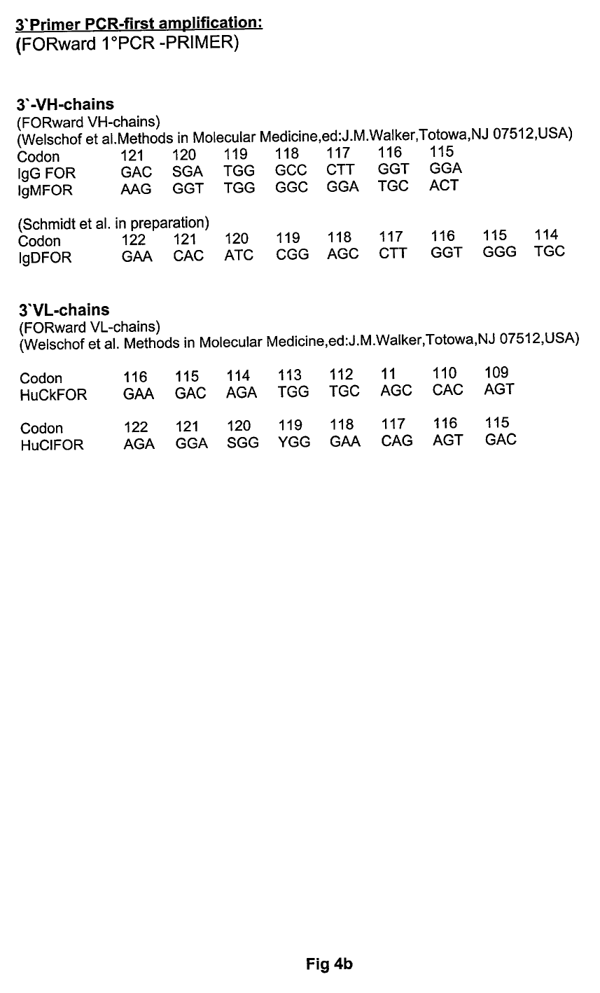

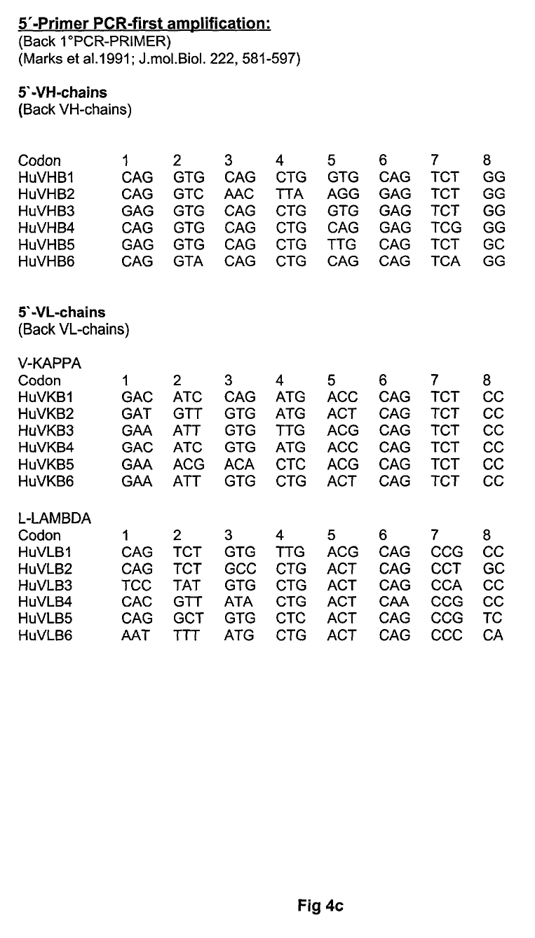

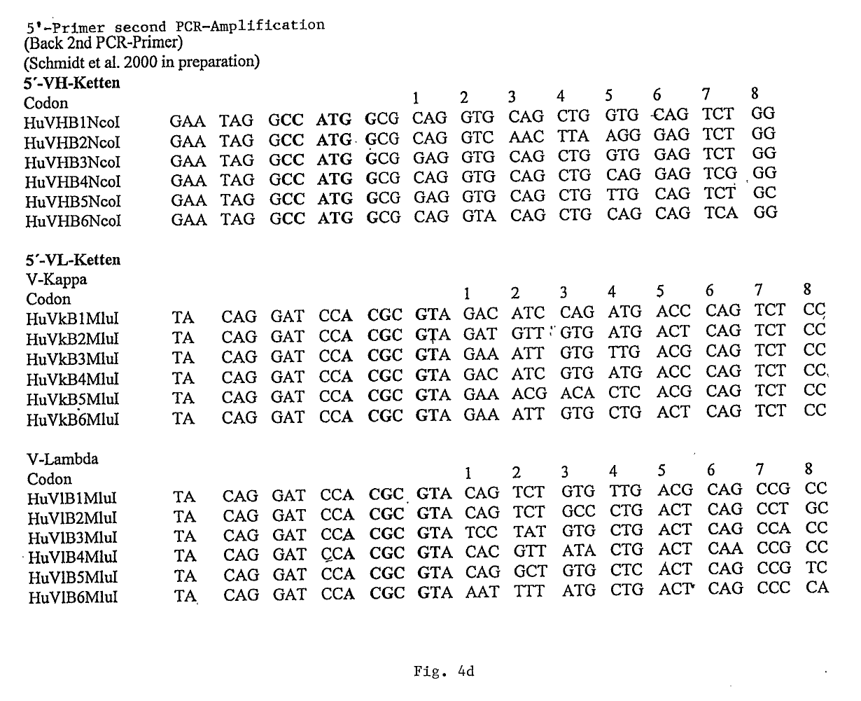

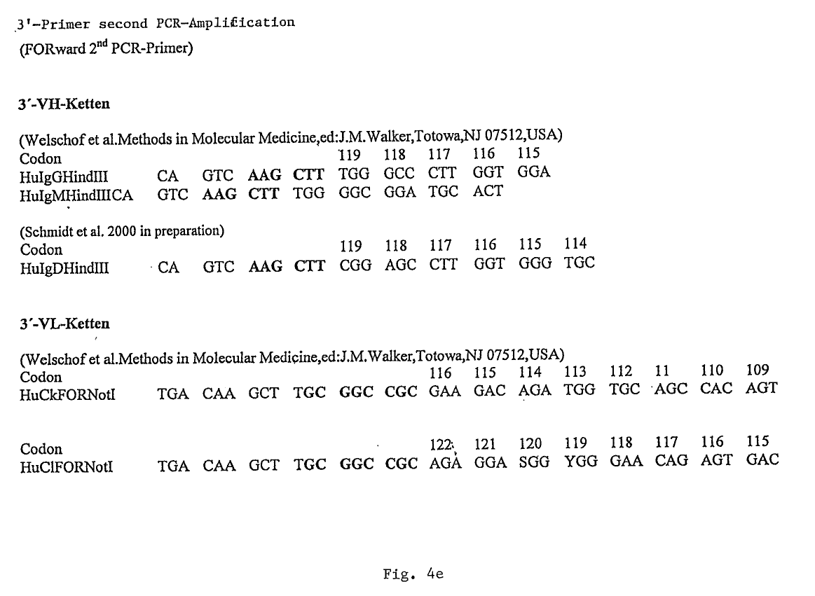

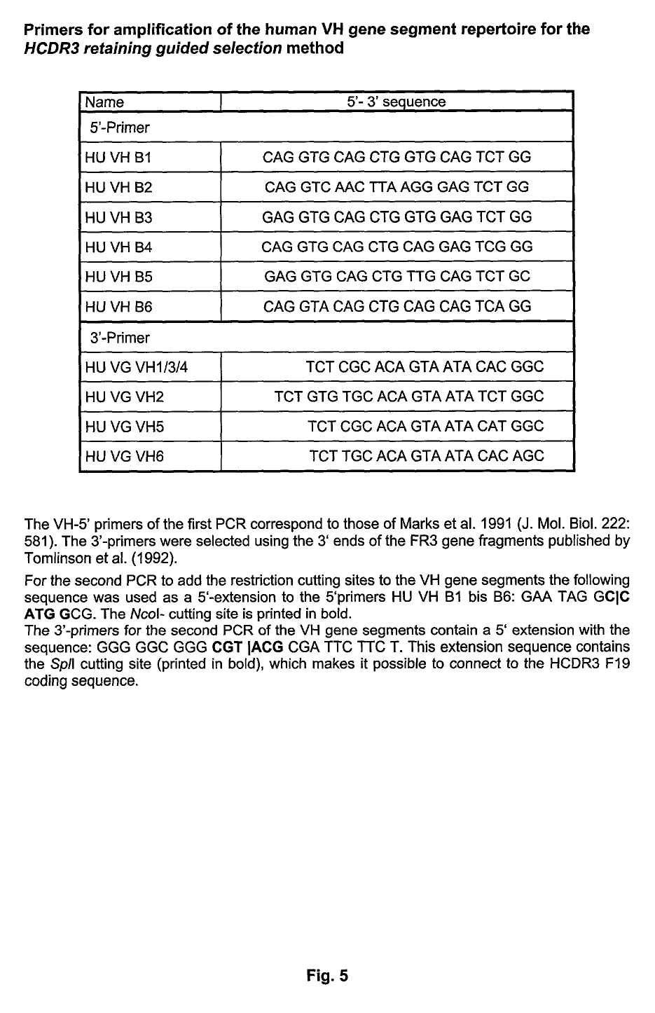

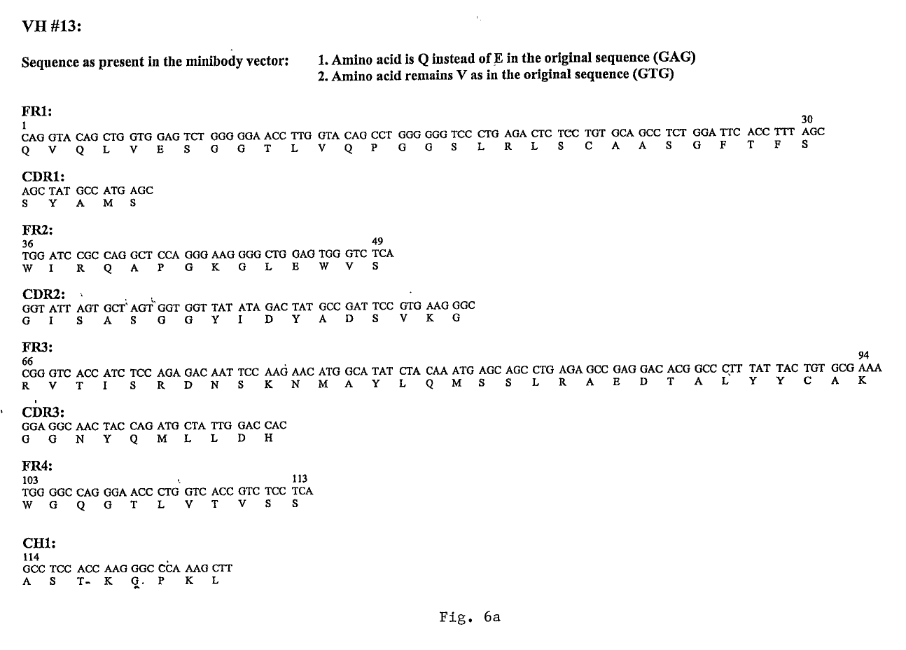

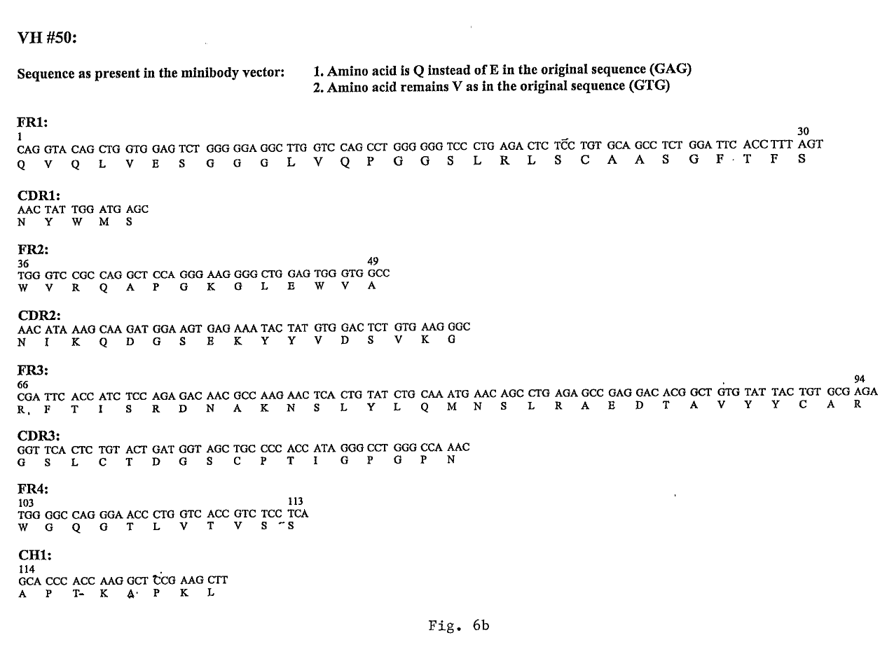

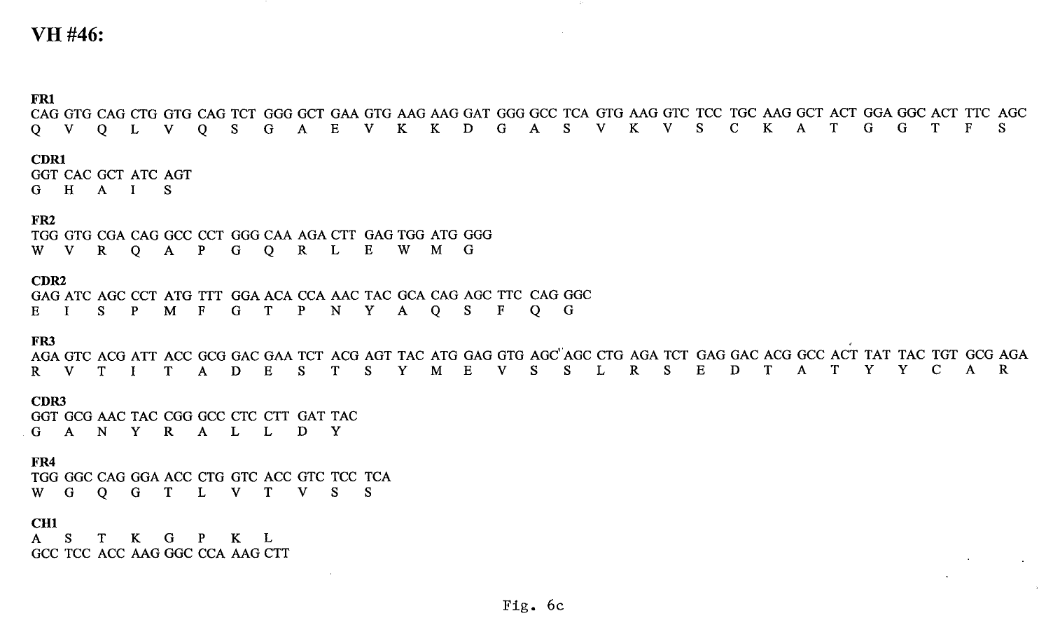

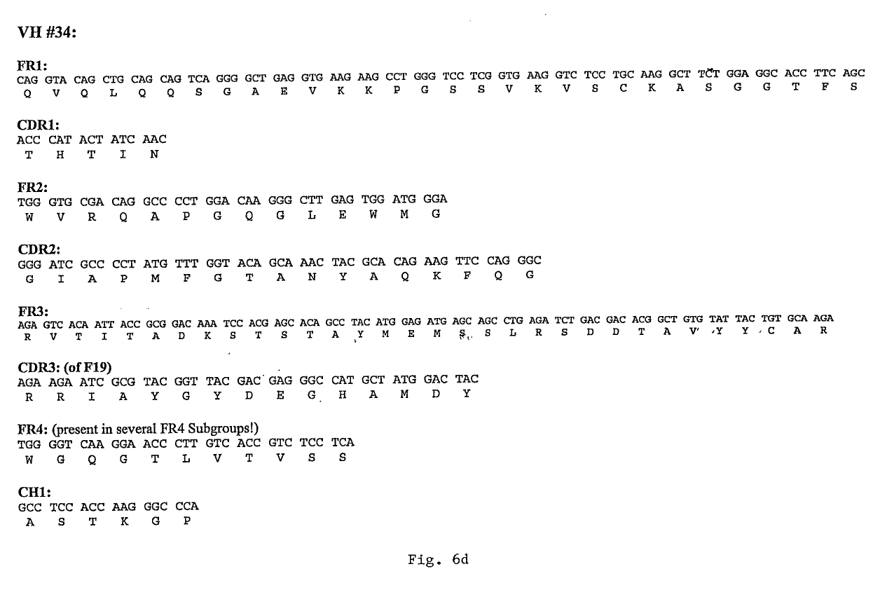

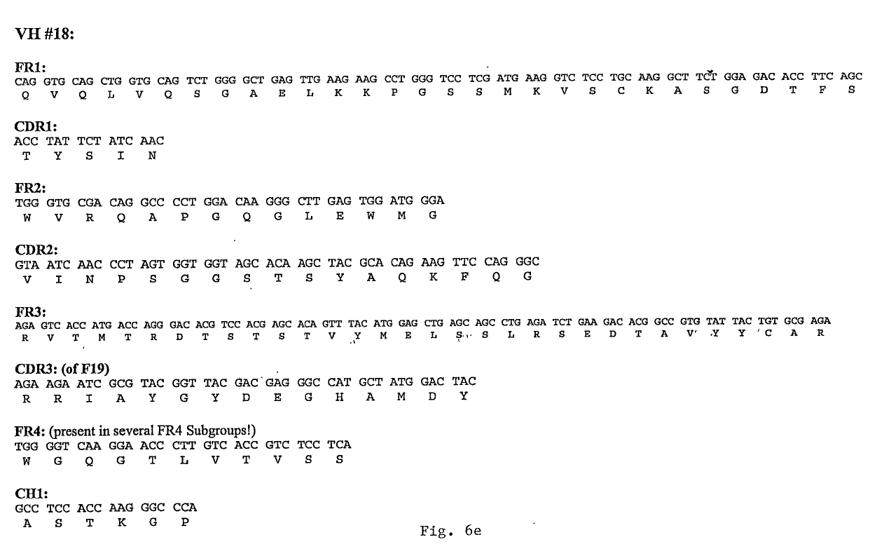

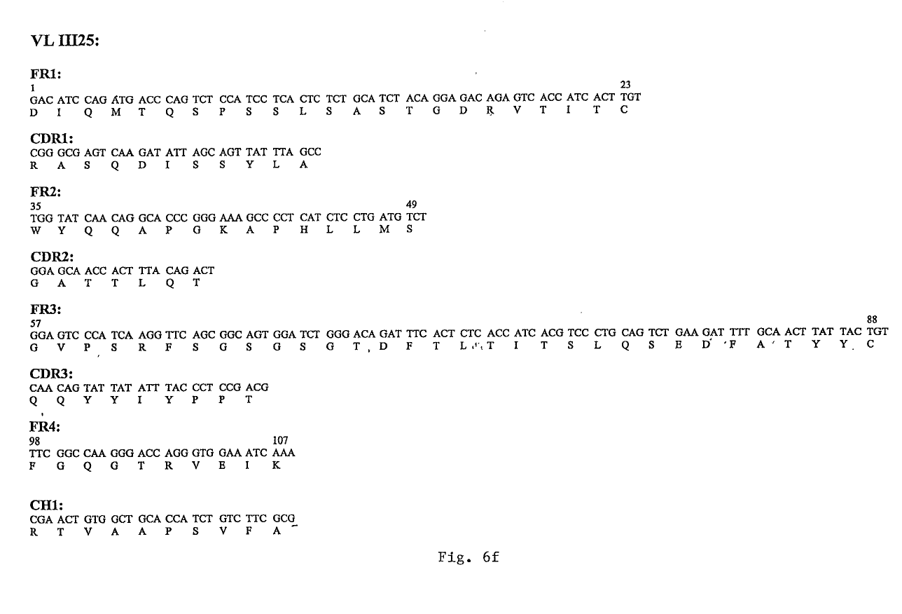

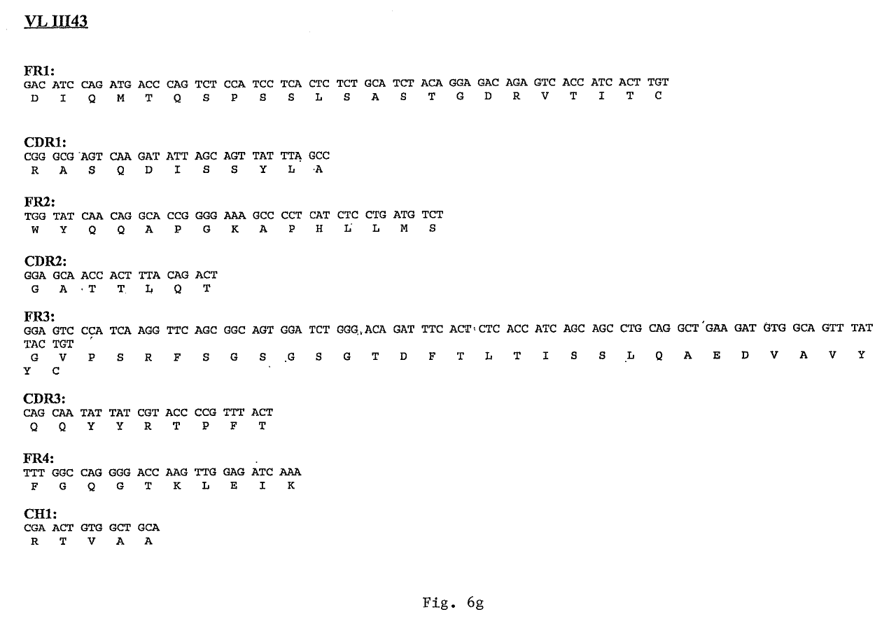

### DESCRIPTION OF THE INVENTION

The problem was solved within the scope of the claims and specification of the present invention. The use of the singular or plural in the claims or specification is in no way intended to be limiting and also includes the other form.

The invention relates to new human or humanised antibody proteins which specifically bind to fibroblast activating protein alpha (FAP), and are either completely human or contain not more than one murine complementarity-determining region (CDR region) of the monoclonal antibody F19 (ATCC accession number HB 8269). The antibodies according to the invention have the surprisingly advantageous property of having a significantly reduced xenoantigenic potential and consequently being better suited for use in humans than the antibodies known from the prior art (cf. also description of the process according to the invention, infra). The antibodies according to the invention advantageously have no or very few parts of the murine amino acid sequence, namely at most one CDR region. The framework regions (FR) of the variable region of the antibodies according to the invention also correspond entirely to human amino acid sequences. In spite of the few murine components, the antibodies according to the invention are nevertheless surprisingly highly specific for the target antigen FAP.

Within the scope of this invention the term antibodies denotes one or more of the polypeptide(s) described in this specification. It also includes human antibody proteins selected from fragments, allelic variants, functional variants, variants based on the degenerative nucleic acid code, fusion proteins with an antibody protein according to the invention, chemical derivatives or a glycosylation variant of the antibody proteins according to the invention.

The preparation methods known from the prior art are unsuitable for obtaining human antibodies according to the invention. With a process according to the invention as hereinafter described and illustrated more fully in the Examples it is possible to obtain a human or humanised antibody according to the invention with reduced xenoantigenic properties. In a preferred preparation process according to the invention the following steps are carried out, for example:

1PCR Amplification of the Human VL-and VH-Repertoires

a) In order to prepare the VH and VL repertoires the various V-gene families are separately amplified with the respective family-specific primers by PCR from cDNA (see Example 1).

b) All Forward/3 ′-primers for VH-and VL-PCR amplification are complementary to the gene sequences of the constant immunoglobulin domains (IgG, IgD, IgM, κ, λ). This enables efficient isotype-specific amplification of the V regions with very few 3 ′-primers. By contrast, in processes known from the prior art a plurality of different 3′-primers complementary to the J-sections of the V regions are used (Marks et al., 1991; J. Mol. Biol. 222:581).

2Preparation and Cloning of a Human VH-Repertoire

In the prior art, up till now, only certain lymphoid tissues have been described with very few different donors as sources of V repertoires (e.g. Vaughan et al., 1996; Nature Biotechnology 14: 309). In order to obtain a human V-repertoire consisting of a large number of clones with high diversity (for details see Example 1) as a basis for the preparation of the antibodies according to the invention, far more different donors are used, i.e. about ten times more than are recommended in the prior art, in non-obvious manner, not only for the lymphoid organs in question, but also the foetal liver and thymus gland are used as a source of V repertoires. Moreover, the IgD repertoire was also amplified, in addition to the IgM and IgG repertoires, in order to achieve great repertoire diversity (see Example 1).

3Preparation of a Combination Repertoire Consisting of a Human VH Repertoire and Various Human FAP-Specific VL Regions

In order to obtain an antibody according to the invention, the VH region known, for example, from the monoclonal, FAP-specific murine antibody F19 may be used and a suitable human FAP-specific VL region may be selected using a guided selection method and a phage display method. Then, using said human VL region as a guiding structure, for example, a human FAP-specific VH region may be selected. The technical problem of the DNA contamination of the combination repertoires with phagemid vectors which code for existing FAP-specific scFv, (e.g. murine scfv from the hybridoma line F19 or the chimeric anti-FAP scfv with human VL and F19 VH) may arise. A guided selection process is described in the Examples.

By combination repertoire is meant the combination, by genetic engineering, of a V repertoire with correspondingly complementary V-sequences. (Complementary with respect to VI to VL and vice versa). The V-sequences used for the combination may consist of one V-sequence, a number of different V-sequences or a V repertoire.

Preferably, an antibody protein according to the invention is characterised in that it comprises a heavy chain (V_{H}) of the immunoglobulin class IgM.

Preferably, an antibody protein according to the invention is also characterised in that it contains a heavy chain (VH) of the class IgG. Non-limiting examples of these are the completely human antibodies scfv #13 and scfv #46 (see Examples).

Preferably, an antibody protein according to the invention is also characterised in that it comprises a heavy chain (VH) of the class IgD. A non-limiting example of this is the human antibody according to the invention scfv #50 (see also Examples). In this antibody the VH-sequence originates from a human IgD and is identical to the germline sequence apart from one amino acid exchange. This advantageously reduces the probability of an allogenic immune response to this VH region in humans.

Preferably, also, an antibody protein according to the invention is characterised in that it comprises a light chain (VL) of the lambda type (λ).

Preferably, also, an antibody protein according to the invention is characterised in that it comprises a light chain (VL) of the kappa type (κ) (see Example, e.g. III25, III43).

For many uses of the antibodies according to the invention it is desirable to have the smallest possible antigen-binding, i.e. FAP-binding units. Therefore in another preferred embodiment an antibody protein according to the invention is a Fab fragment (Fragment antigen-binding=Fab). These FAP-specific antibody proteins according to the invention consist of the variable regions of both chains which are held together by the adjacent constant region. These may be formed by protease digestion, e.g. with papain, from conventional antibodies, but similar Fab fragments may also be produced in the mean time by genetic engineering. In another preferred embodiment an antibody protein according to the invention is an F(ab′)2 fragment, which may be prepared by proteolytic cleaving with pepsin.

Using genetic engineering methods it is possible to produce shortened antibody fragments which consist only of the variable regions of the heavy (VH) and of the light chain (VL). These are referred to as Fv fragments (fragment of the variable part). In another preferred embodiment, an FAP-specific antibody molecule according to the invention is such an Fv fragment. Since these Fv-fragments lack the covalent bonding of the two chains by the cysteines of the constant chains, the Fv fragments are often stabilised. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins of this kind known from the prior art are described in Huston et al. (1988, PNAS 16: 5879-5883). Therefore, in another preferred embodiment an FAP-specific antibody protein according to the invention is a single-chain-Fv protein (scFv).

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucin-zipper domains. However, there are also strategies in which the interaction between the VHJVL regions of the scFv are used for the multimerisation (e.g. di-, tri-and pentabodies). Therefore in another preferred embodiment an antibody protein according to the invention is an FAP-specific diabody antibody fragment. By diabody the skilled person means a bivalent homodimeric scFv derivative (Hu et al., 1996, PNAS 16: 5879-5883). The shortening of the Linker in an scFv molecule to 5-10 amino acids leads to the formation of homodimers in which an inter-chain VHNVL-superimposition takes place. Diabodies may additionally be stabilised by the incorporation of disulphide bridges. Examples of diabody-antibody proteins from the prior art can be found in Perisic et al. (1994, Structure 2: 1217-1226).

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a Linker region. The disulphide bridges in the Hinge region are mostly formed in higher cells and not in prokaryotes. In another preferred embodiment an antibody protein according to the invention is an FAP-specific minibody antibody fragment. Examples of minibody-antibody proteins from the prior art can be found in Hu et al. (1996, Cancer Res. 56: 3055-61). By triabody the skilled person means a: trivalent homotrimeric scFv derivative (Kortt et al. 1997 Protein Engineering 10: 423-433). ScFv derivatives wherein VH-VL are fused directly without a linker sequence lead to the formation of trimers.

The skilled person will also be familiar with so-called miniantibodies which have a bi-, tri-or tetravalent structure and are derived from scFv. The multimerisation is carried out by di-, tri-or tetrameric coiled coil structures (Pack et al., 1993 Biotechnology 11:, 1271-1277; Lovejoy et al. 1993 Science 259: 1288-1293; Pack et al., 1995 J. Mol. Biol. 246: 28-34).

Therefore, in another preferred embodiment an antibody protein according to the invention is an FAP-specific multimerised molecule based on the abovementioned antibody fragments and may be, for example, a triabody, a tetravalent miniantibody or a pentabody.

Particularly preferred, an antibody protein according to the invention is totally human. Another preferred antibody protein according to the invention is characterised in that the variable region of the heavy chain (V_{H}) contains the amino acid sequence according to SEQ ID NO: 1 (VH13).

Another preferred antibody protein according to the invention is characterised in that the variable region of the heavy chain (V_{H}) contains the amino acid sequence according to SEQ ID NO:2 (VH46).

Another preferred antibody protein according to the invention is characterised in that the variable region of the heavy chain (V_{H}) contains the amino acid sequence according to SEQ ID NO:3 lo (VH50).

Another preferred antibody protein according to the invention is characterised in that the variable region of the light chain (V_{L}) contains the amino acid sequence according to SEQ ID NO:4 (VLIII25).

Another preferred antibody protein according to the invention is characterised in that the variable region of the heavy chain (V_{H}) is coded by the nucleotide sequence according to SEQ ID NO:5 (VH 13) or by fragments or degenerate variants thereof

Another preferred antibody protein according to the invention is characterised in that the variable region of the heavy chain (V_{H}) is coded by the nucleotide sequence according to SEQ ID NO:6 (VH46) or by fragments or degenerate variants thereof.

Another preferred antibody protein according to the invention is characterised in that the variable region of the heavy chain (V_{H}) is coded by the nucleotide sequence according to SEQ ID NO:7 (VH50) or by fragments or degenerate variants thereof.

Another preferred antibody protein according to the invention is characterised in that the variable region of the light chain (V_{L}) is coded by the nucleotide sequence according to SEQ ID NO:8 (VLIII25) or by fragments or degenerate variants thereof.

An especially preferred antibody protein according to the invention is characterised in that the variable region of the heavy chain (V_{H}) contains the amino acid sequence according to SEQ ID NO: 1 (VH13) and the variable region of the light chain (V_{L}) contains the amino acid sequence according to SEQ ID NO:4 (VLIII25).

Another particularly preferred antibody protein according to the invention is characterised in that the coding sequence of the variable region of the heavy chain (V_{H}) contains the nucleotide sequence according to SEQ ID NO:5 (VH13) and the coding sequence of the variable region of the light chain (V_{L}) contains the nucleotide sequence according to SEQ ID NO:8 (VLIII25).

Another particularly preferred antibody protein according to the invention is characterised in that the variable region of the heavy chain (V_{H}) contains the amino acid sequence according to SEQ ID NO:2 (VH46) and the variable region of the light chain (V_{L}) contains the amino acid sequence according to SEQ ID NO:4 (VLIII25).

Another particularly preferred antibody protein according to the invention is characterised in that the coding sequence of the variable region of the heavy chain (V_{H}) contains the nucleotide sequence according to SEQ ID NO:6 (VH46) and the coding sequence of the variable region of the light chain (V_{L}) contains the nucleotide sequence according to SEQ ID NO:8 (VLIII25).

Another particularly preferred antibody protein according to the invention is characterised in that the variable region of the heavy chain (V_{H}) contains the amino acid sequence according to SEQ ID NO:3 (VH50) and the variable region of the light chain (V_{L}) contains the amino acid sequence according to SEQ ID NO:4 (VLIII25).

Another particularly preferred antibody protein according to the invention is characterised in that the coding sequence of the variable region of the heavy chain (V_{H}) contains the nucleotide sequence according to SEQ ID NO:7 (VH50) and the coding sequence of the variable region of the light chain (V_{L}) contains the nucleotide sequence according to SEQ ID NO:8 (VLIII25).

Particularly preferred, an antibody protein according to the invention is humanised. The humanised antibody protein according to the invention has the advantage, over the FAP-specific antibody proteins known from the prior art, that it does not contain all six murine CDR regions of F19, but only one murine CDR region, as described in the following preferred embodiments. This antibody protein according to the invention advantageously has a lesser xenoantigenic potential than the antibody proteins known from the prior art. Surprisingly, the inventors have succeeded in producing antibody molecules which contain only one murine CDR region, against the prevailing opinion that at least two murine CDR regions are necessary for successful humanisation (Rader et al, 1998, Proc. Natl. Acad. Sci. USA, 95: 8910).

Another surprising property in the case of humanised scFv 34 and scFv 18 is that these scFv exhibit a higher apparent binding affinity for FAP+-cells (EC₅₀ 6 nM) than the FAP-specific antibodies such as e.g. scFv F19 (EC₅₀20 nM) known from the prior art.

A preferred process according to the invention for preparing humanised antibodies according to the invention may be described by the following steps, for example:

1Humanisation of scFv F19 by the HCDR3 retaining Guided selection method

Our experience has shown that by using the “Guided selection” process, human antibody (Ab) can be selected which have a different epitope specificity from the parental murine Ab. In order to overcome this disadvantage in the prior art, the HCDR3 F 19 was advantageously retained in the Guided selection process for humanising scFv F19 as well as in the final humanised product. The prior art (Rader et al., 1998, PNAS 95: 8910) describes only antibodies humanised by Guided selection in which both the LCDR3 and also the HCDR3 of the parental murine Ab are retained (see Example 1).

2Combination of a human VH-gene segment repertoire with murine HCDR3 F19

The VH segments of all known human VH families are to be combined with HCDR3 F 19 in order to generate as complex a combination repertoire as possible. Advantageously, this is preferably done, e.g., by integrating a cutting site for the restriction enzyme Pfl23II in the HCDR3 F 19 without altering the coding at the amino acid level. For combining the PCR-amplified human VH-gene segments, a Phage display vector was developed which contains the following Ab-sequence sections: HCDR3 F19 with a Pfl23II cutting site, a human VH FR4 region with high homology with the corresponding region from F19 as well as various selected human anti-FAP VL regions (see the diagram in Example 1). The primers for PCR amplification of the VH-gene segment repertoires are shown in Example 1.

This preferred process has the following advantages over the prior art for combining VH-gene segment repertoires with defined CDR3 regions:

Schier et al. 1996: J. Mol. Biol. 255: 28 In this prior art a restriction cutting site (BssHII) was integrated in the 3′ region of VH FR 3. The incorporation of this cutting site via PCR is, however, connected with an altered amino acid sequence in various VH-gene families. For this reason, in Schier et al. Only some of the VH-gene families were able to be included in the combination repertoire.

PCR overlap extension Rader et al. 1998

This process does indeed make it possible to include all VH-gene families in the combination, but the disadvantages are a low linking efficiency and a high error rate. This increases the probability of inactive scFv mutants and especially clones with an interrupted scFv reading frame, leading to genetically unstable combination repertoires.

Use of different human FAP-specific VL regions as a guide structure

In order to increase the probability of selecting an ScFv analogous to F19, the human VH repertoire (see 2) was combined with the sequences of different human FAP-specific VL regions. (Carried out analogously to human antibodies, supra).

Stringent washing step in Phage display selection

This procedure was used to eliminate low-affinity and polyreactive antibodies during the selection process (for method see below).

5Use of an efficient screening process for identifying the selected humanised scFv

During the HCDR3 retaining guided selection process a very large number of clones were concentrated. The scFv #34 and #18 can advantageously be identified by the screening process described in Mersmann et al. 1998 (J. Immunol. Methods, 220: 51).

Another preferred antibody protein according to the invention is characterised in that it contains murine CDR 1 of the light chain (V_{L}) of the monoclonal antibody F19.

Another preferred antibody protein according to the invention is characterised in that it contains murine CDR 2 of the light chain (V_{L}) of the monoclonal antibody F19.

Another preferred antibody protein according to the invention is characterised in that it contains murine CDR 3 of the light chain (V_{L}) of the monoclonal antibody F19.

Another preferred antibody protein according to the invention is characterised in that it contains murine CDR 1 of the heavy chain (V_{H}) of the monoclonal antibody F19.

Another preferred antibody protein according to the invention is characterised in that it contains murine CDR 2 of the heavy chain (V_{H}) of the monoclonal antibody F19.

Another preferred antibody protein according to the invention is characterised in that it contains murine CDR 3 of the heavy chain (V_{H}) of the monoclonal antibody F19.

Another preferred antibody protein according to the invention is characterised in that the variable region of the heavy chain (V_{H}) contains the amino acid sequence according to SEQ ID NO:9 (VH34).

Another preferred antibody protein according to the invention is characterised in that the variable region of the heavy chain (V_{H}) contains the amino acid sequence according to SEQ ID NO: 10 (VH18).

Another preferred antibody protein according to the invention is characterised in that the variable region of the light chain (V_{L}) contains the amino acid sequence according to SEQ ID NO: 11 (VLIII43).

Another preferred antibody protein according to the invention is characterised in that the variable region of the heavy chain (V_{H}) is coded by the nucleotide sequence according to SEQ ID NO: 12 (VH34) or by fragments or degenerate variants thereof.

Another preferred antibody protein according to the invention is characterised in that the variable region of the heavy chain (V_{H}) is coded by the nucleotide sequence according to SEQ ID NO: 13 (VH18) or by fragments or degenerate variants thereof.

Another preferred antibody protein according to the invention is characterised in that the variable region of the light chain (V_{L}) is coded by the nucleotide sequence according to SEQ ID NO: 14 (VLIII43) or by fragments or degenerate variants thereof.

An especially preferred antibody protein according to the invention is characterised in that the variable region of the heavy chain (V_{H}) contains the amino acid sequence according to SEQ ID NO:9 (VH34) and the variable region of the light chain (V_{L}) contains the amino acid sequence according to SEQ ID NO: 11 (VLIII43).

Another particularly preferred antibody protein according to the invention is characterised in that the coding sequence of the variable region of the heavy chain (V_{H}) contains the nucleotide sequence according to SEQ ID NO: 12 (VH34) and the coding sequence of the variable region of the light chain (V_{L}) contains the nucleotide sequence according to SEQ ID NO: 14 (VLIII43).

Another particularly preferred antibody protein according to the invention is characterised in that the variable region of the heavy chain (V_{H}) contains the amino acid sequence according to SEQ ID NO:10 (VHI 18) and the variable region of the light chain (V_{L}) contains the amino acid sequence according to SEQ ID NO: 11 (VLIII43).

Another particularly preferred antibody protein according to the invention is characterised in that the coding sequence of the variable region of the heavy chain (V_{H}) contains the nucleotide sequence according to SEQ ID NO:13 (VH18) and the coding sequence of the variable region of the light chain V_{L}) contains the nucleotide sequence according to SEQ ID NO: 14 (VLIII43).

Another preferred embodiment of the invention comprises a nucleic acid which codes for an antibody protein according to the invention. Preferably, too, a nucleic acid according to the invention is characterised in that it contains 5′ or 3′ or 5′ and 3′ untranslated regions. The nucleic acid according to the invention may contain other untranslated regions upstream and/or downstream. The untranslated region may contain a regulatory element, such as e.g. a transcription initiation unit (promoter) or enhancer. Said promoter may, for example, be a constitutive, inducible or development-controlled promoter. Preferably, without ruling out other known promoters, the promoters may include the constitutive promoters of the human Cytomegalovirus (CMV) and Rous sarcoma virus (RSV), as well as the Simian virus 40 (SV40) and Herpes simplex promoter. Inducible promoters according to the invention comprise antibiotic-resistant promoters, heat-shock promoters, hormone-inducible “Mammary tumour virus promoter” and the metallothioneine promoter. Preferably, too, a nucleic acid according to the invention is characterised in that it codes for a fragment of the antibody protein according to the invention. This refers to part of the polypeptide according to the invention.

Preferably, too, a nucleic acid according to the invention is characterised in that it codes for a functional variant of the antibody protein according to the invention. This denotes polypeptides which are largely identical to an antibody protein according to the invention and which have the same biological activity as an antibody protein according to the invention or have an inhibiting effect on an antibody protein according to the invention. A variant of an antibody protein according to the invention may differ from an antibody protein according to the invention by substitution, deletion or addition of one or more amino acids, preferably by 1 to 10 amino acids.

Preferably, too, a nucleic acid according to the invention is characterised in that it codes for an allelic variant of the antibody protein according to the invention. Preferably, too, a nucleic acid according to the invention is characterised in that it codes for variants of the antibody protein according to the inventions on the basis of the degenerative code of the nucleic acids. Preferably, too, a nucleic acid is characterised in that it is able to hybridise to a nucleic acid according to the invention under stringent conditions. Stringent conditions are known to those skilled in the art and are found particularly in Sambrook et al. (1989). Molecular Cloning: A Laboratory Manual, ₂^{nd} ed., Cold Spring Harbor Laboratory Press, Cold Spring Harbor, N.Y.

Another particularly preferred embodiment of the invention comprises an antibody protein, characterised in that it contains an amino acid sequence according to SEQ ID NO:15 or a part thereof or a functional variant thereof.

Another particularly preferred embodiment of the invention comprises an antibody protein, characterised in that it contains an amino acid sequence according to SEQ ID NO:16 or a part thereof or a functional variant thereof.

Another particularly preferred embodiment of the invention comprises an antibody protein, characterised in that it contains an amino acid sequence according to SEQ ID NO:17 or a part thereof or a functional variant thereof.

Another particularly preferred embodiment of the invention comprises an antibody protein, characterised in that it contains an amino acid sequence according to SEQ ID NO:18 or a part thereof or a functional variant thereof.

Another particularly preferred embodiment of the invention comprises an antibody protein, characterised in that it contains an amino acid sequence according to SEQ ID NO: 19 or a part thereof or a functional variant thereof.

Another particularly preferred embodiment of the invention comprises an antibody protein, characterised in that it is coded by a nucleotide sequence according to SEQ ID NO:20 or a part thereof or a functional variant thereof.

Another particularly preferred embodiment of the invention comprises an antibody protein, characterised in that it is coded by a nucleotide sequence according to SEQ ID NO:21 or a part thereof or a functional variant thereof.

Another particularly preferred embodiment of the invention comprises an antibody protein, characterised in that it is coded by a nucleotide sequence according to SEQ ID NO:22 or a part thereof or a functional variant thereof.

Another particularly preferred embodiment of the invention comprises an antibody protein, characterised in that it is coded by a nucleotide sequence according to SEQ ID NO:23 or a part thereof or a functional variant thereof.

Another particularly preferred embodiment of the invention comprises an antibody protein, characterised in that it is coded by a nucleotide sequence according to SEQ ID NO:24 or a part thereof or a functional variant thereof.

Another particularly preferred embodiment of the invention comprises an antibody protein, characterised in that it corresponds to the amino acid sequence according to SEQ ID NO:15.

Another particularly preferred embodiment of the invention comprises an antibody protein, characterised in that it corresponds to the amino acid sequence according to SEQ ID NO:16.

Another particularly preferred embodiment of the invention comprises an antibody protein, characterised in that it corresponds to the amino acid sequence according to SEQ ID NO:17.

Another particularly preferred embodiment of the invention comprises an antibody protein, characterised in that it corresponds to the amino acid sequence according to SEQ ID NO:18.

Another particularly preferred embodiment of the invention comprises an antibody protein, characterised in that it corresponds to the amino acid sequence according to SEQ ID NO:19.

Another particularly preferred embodiment of the invention comprises an antibody protein, characterised in that it is coded by the nucleotide sequence according to SEQ ID NO:20.

Another particularly preferred embodiment of the invention comprises an antibody protein, characterised in that it is coded by the nucleotide sequence according to SEQ ID NO:21.

Another particularly preferred embodiment of the invention comprises an antibody protein, characterised in that it is coded by the nucleotide sequence according to SEQ ID NO:22.

Another particularly preferred embodiment of the invention comprises an antibody protein, characterised in that it is coded by the nucleotide sequence according to SEQ ID NO:23.

Another particularly preferred embodiment of the invention comprises an antibody protein, characterised in that it is coded by the nucleotide sequence according to SEQ ID NO:24.

Sequence ID NO:refers to the number specified under <400> in the Sequence Listing, so that e.g. the nucleotide sequence according to SEQ ID NO:24 is listed as <400 > 24.

Another aspect of the present invention relates to a recombinant DNA vector which contains a nucleic acid according to the invention. Examples are viral vectors such as e.g. Vaccinia, Semliki-Forest-Virus and Adenovirus. Vectors for use in COS-cells have the SV40 origin of replication and make it possible to achieve high copy numbers of the plasmids. Vectors for use in insect cells are, for example, E. coli transfer vectors and contain e.g. the DNA coding for polyhedrin as promoter.

Another aspect of the present invention relates to a recombinant DNA vector according to the invention which is an expression vector.

Yet another aspect of the present invention is a host which contains a vector according to the invention.

Another host according to the invention is a eukaryotic host cell. The eukaryotic host cells according to the invention include fungi, such as e.g. Pichia pastoris, Saccharomyces cerevisiae, Schizosaccharomyces, Trichoderma, insect cells (e.g. from Spodoptera frugiperda Sf-9, with a Baculovirus expression system), plant cells, e.g. from Nicotiana tabacum, mammalian cells, e.g. COS cells, BHK, CHO or myeloma cells.

In descendants of the cells of the immune system in which antibody proteins are also formed in our body, the antibody proteins according to the invention are particularly well-folded and glycosylated.

Therefore, a preferred host according to the invention is a mammalian cell.

Particularly preferred, a host according to the invention is a BHK, CHO or COS cell.

Another host according to the invention is a bacteriophage.

Another host according to the invention is a prokaryotic host cell. Examples of prokaryotic host cells are Escherichia coli, Bacillus subtilis, Streptomyces or Proteus mirabilis.

The invention relates to a process for preparing antibody protein according to the invention, which comprises the following steps: a host according to the invention as described above is cultivated under conditions in which said antibody protein is expressed by said host cell and said antibody protein is isolated.

The antibody proteins according to the invention may be expressed in any of the hosts described above.

Preparation with prokaryotic expression systems such as Escherichia coli, Bacillus subtilis, Streptomyces or Proteus mirabilis is especially suitable for antibody fragments according to the invention, such as Fab-, F(ab′)2-, scFv fragments, minibodies, diabodies and multimers of said fragments. The antibody proteins according to the invention are prepared by a process according to the invention either intracellularly, e.g. in inclusion bodies, by secretion into bacteria with no cell walls such as, for example, Proteus mirabilis or by periplasmatic secretion into Gram-negative bacteria using suitable vectors for this purpose. In Example 2, the preparation of the antibody proteins according to the invention in prokaryotes is described by way of example. Examples from the prior art for the preparation of scFv-antibody proteins are described in Rippmann et al. (1998, Appl. Environ. Microbiol., 1998, 64: 4862-4869). Other examples are known to those skilled in the art.

The antibody proteins according to the invention may also be prepared in a process according to the invention in fungi, such as e.g. Pichia pastoris, Saccharomyces cerevisiae, Schizosaccharomyces, Trichoderma with vectors which lead to intracellular expression or secretion.

The process according to the invention for preparing the antibody proteins may also be carried out with insect cells, e.g. as a transient or stabile expression system or Baculovirus expression system.

Here, Sf-9 insect cells, for example, are infected with e.g. Autographa californica nuclear polyhedrosis virus (AcNPV) or related viruses. There is no risk of contamination with viruses which are pathogenic to mammals, therefore therapeutic antibodies according to the invention may also advantageously be prepared in insect cells. The E. coli transfer vectors described above contain, for example, as promoters, the DNA which codes for polyhedrin, behind which the DNA coding for the antibodies according to the invention is cloned. After identification of a correct transfer vector clone in E. coli, this is transfected together with incomplete Baculovirus DNA into an insect cell and recombined with the Baculovirus DNA so as to form viable Baculoviruses. Using powerful insect cell promoters, in a process according to the invention, large amounts of the antibody protein according to the invention are formed which are secreted into the medium e.g. by fusion with eukaryotic signal sequences. Insect cell expression systems for die expression of antibody proteins are commercially obtainable. Insect cell expression systems are particularly suitable for the scFv fragments according to the invention and Fab or F(ab′)2 fragments and antibody proteins or fragments thereof which are fused with effector molecules, but are also suitable for complete antibody molecules.

One advantage of mammalian expression systems is that they give rise to very good glycosylation and folding conditions, e.g. transient expression systems, e.g. in COS-cells or stable expression systems e.g. BHK, CHO, myeloma cells (cf. also Example 2). Mammalian cells may also be used, for example, with viral expression systems e.g. Vaccinia, Semliki-Forest-Virus and Adenovirus. Transgenic animals such as cows, goats and mice are also suitable for a process according to the invention. Transgenic plants such as Nicotiana tabacum (tobacco) may also be used in a process according to the invention. They are particularly suitable for the preparation of antibody fragments according to the invention. After genomic integration of the nucleic acid according to the invention which codes for an antibody protein according to the invention which is fused to a signal sequence, secretion of the antibody protein into the interstitial space can be achieved.

The invention relates in particular to a process according to the invention wherein said host is a mammalian cell, preferably a CHO or COS cell.

The invention relates in particular to a process according to the invention wherein said host cell is co-transfected with two plasmids which carry the expression units for the light or the heavy chain. The antibody proteins of the present invention are highly-specific agents for guiding therapeutic agents to the FAP antigen. Therefore, another preferred antibody protein according to the invention is characterised in that said antibody protein is coupled to a therapeutic agent.

This antibody protein according to the invention may, preferably, be coupled to a therapeutic agent or an effector molecule by genetic engineering. According to the invention, a therapeutic agent of this kind includes cytokines, such as for example interleukins (IL) such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumour necrosis factor (TNF) TNF alpha and TNF beta, TRAIL, an immunomodulatory or immunostimulant protein, or an apoptosis-or necrosis-inducing protein. Therefore, the antibody-effector molecule conjugates according to the invention comprise antibody-cytokine fusion proteins, and also bispecific antibody derivatives and antibody-superantigen fusion proteins. These are preferably used for activating the body's own anti-tumoral defense mechanisms and are thus suitable for therapeutic use. Another preferred FAP-specific antibody protein according to the invention is characterised in that it is used for somatic gene therapy. For example, this may be achieved by use as an antibody toxin-fusion protein (as described for example in Chen et al. 1997, Nature 385: 78-80 for other targets) or as a fusion protein consisting of an antibody according to the invention and a T-cell receptor or Fc-receptor (transmembrane and intracellular region, cf. e.g. Wels et al., 1995, Gene, 159: 73-80). The use for somatic gene therapy may also be carried out by expression of the nucleic acid according to the invention in a shuttle vector, a gene probe or a host cell.

Another preferred antibody protein coupled to a therapeutic according to the invention is characterised in that said therapeutic agent is selected from among the radioisotopes, toxins or immunotoxins, toxoids, fusion proteins, for example, genetically engineered fusion proteins, inflammatory agents and chemotherapeutic agents and elements which allow a neutron capturing reaction, such as e.g. boron (boron-neutron capturing reaction, BNC).

Another preferred antibody protein coupled to a radioisoptope according to the invention is characterised in that said radioisotope is a β-emitting radioisotope.

Another preferred antibody protein coupled to a radioisoptope according to the invention is characterised in that said radioisotope is selected from among ¹⁸⁶rhenium, ¹⁸⁸rhenium, ¹³¹iodine and ⁹⁰yttrium which have proved particularly suitable for linking to the antibodies according to the invention as therapeutic agents. A process for radio-iodine labelling of the antibodies according to the invention is described in WO 93/05804.

Another preferred antibody protein according to the invention is characterised in that it is labelled.

Another preferred antibody protein according to the invention is characterised in that it is labelled with a detectable marker.

Another preferred antibody protein according to the invention is characterised in that the detectable marker is selected from among the enzymes, dyes, radioisotopes, digoxygenine, streptavidine and biotin.

Another preferred antibody protein according to the invention is characterised in that it is coupled to an imageable agent.

Another preferred antibody protein according to the invention is characterised in that it is coupled to an imageable agent which is a radioisotope.

Another preferred antibody protein according to the invention is characterised in that it is coupled to a radioisotope wherein said radioisotope is a β-emitting radioisotope.

Another preferred antibody protein according to the invention is characterised in that it is coupled to a radioisotope wherein said radioisotope is ¹²⁵iodine.

Another important aspect of the present invention relates to a pharmaceutical preparation which contains an antibody protein according to the invention and one or more pharmaceutically acceptable carrier substances. Pharmaceutically acceptable carriers or adjuvants in this invention may be, for example, physiologically acceptable compounds which stabilise or improve the absorption of antibody protein according to the invention. Such physiologically acceptable compounds include, for example, carbohydrates such as glucose, sucrose or dextrane, antioxidants such as ascorbic acid or glutathione, chelating agents, lower-molecular compounds or other stabilisers or adjuvants (see also Remington's Pharmaceutical Sciences, 18th Edition, Mack Publ., Easton.). The skilled person knows that the choice of a pharmaceutically acceptable carrier depends, for example, on the route of administration of the compound. The said pharmaceutical composition may also contain a vector according to the invention for gene therapy and may additionally contain, as adjuvant, a colloidal dispersion system or liposomes for targeted administration of the pharmaceutical composition. A host or a host cell which contains a vector according to the invention may also be used in a pharmaceutical composition within the scope of this invention, for example, for gene therapy.

Another important aspect of the present invention relates to the use of a pharmaceutical preparation according to the invention for treating or imaging tumours, wherein said tumours are associated with activated stromal fibroblasts.

This use according to the invention relates particularly to cases wherein said tumours can be categorised as one of the following types of cancer or form the basis thereof and are therefore selected from among colorectal cancer, non-small-cell lung cancer, breast cancer, head and neck cancer, ovarian cancer, lung cancer, bladder cancer, pancreatic cancer and metastatic brain cancer. Yet another important aspect of the present invention relates to the use of an antibody protein according to the invention for preparing a pharmaceutical preparation for treating cancer. Yet another important aspect of the present invention relates to the use of an antibody protein according to the invention for imaging activated stromal fibroblasts.

An additional aspect of the present invention is a process for detecting activated stromal fibroblasts in wound healing, inflammatory processes or in a tumour which is characterised in that a probe, which might possibly contain activated fibroblasts, is contacted with an antibody protein according to the invention under conditions which are suitable for forming a complex from said antibody protein with its antigen and the formation of said complex and hence the presence of activated stromal fibroblasts in wound healing, inflammatory processes or in a tumour is detected.

The process according to the invention described in the previous paragraph is particularly characterised in that said tumour is selected from among colorectal cancer, non-small-cell lung cancer, breast cancer, head and neck cancer, ovarian cancer, lung cancer, bladder cancer, pancreatic cancer and metastatic brain cancer.

The invention further includes a process for detecting tumour stroma wherein a suitable probe is so contacted with an antibody protein according to the invention under suitable conditions for the formation of an antibody-antigen complex, the complex thus formed is detected and the presence of the complex thus formed is correlated with the presence of tumour stroma.

The process according to the invention described in the previous paragraph is particularly characterised in that said antibody is labelled with a detectable marker. p The following Examples are intended to aid the understanding of the invention and should in no way be regarded as limiting the scope of the invention.

### EXAMPLE 1

### 1 Cloning Of A Human VH Repertoire For The Guided Selection Method

A) Development of Anti-FAP Antibodies With Fully Human V Regions

Method of preparation:

1. Cloning of F19VH

2. Preparation of human V-repertoire

Reverse transcription, PCR amplification of human VL (λ, κ) repertoires from peripheral blood lymphocytes, an improved process according to Persson et al. 1991, PNAS 88: 2432.

Cloning the VL repertoires in Phage display vector (pSEX81, DKFZ, Heidelberg; Breitling et al., 1991, Gene 104:147) size of repertoire: VL 10⁷ clones

Reverse transcription, PCR amplification of human VH repertoire (IgG, IgD, IgM) from peripheral blood lymphocytes, thymus gland, spleen, bone marrow, tonsils, lymph nodes, foetal liver (improved according to Persson et al. 1991, PNAS 88: 2432)

Improvement of process: Use of IgD and different lymphoid tissue

Cloning the VH repertoire in Phage display vector (pSEX81, DKFZ, Heidelberg; Breitling et al., 1991, Gene 104:147) size of repertoire: VH 3×10⁸ clones

3. Selection of human VL regions which functionally replace VL F19:

Phage display selection and Guided selection strategy with VH F19 as the guiding structure (improved according to McCafferty et al., 1990, Nature 348: 552 and Jespers et al., 1994, Bio/Technology 12:899) Isolation of human FAP-specific VL regions (known as VL:III5, III10, III25, III43)

4. Selection of human VH regions which functionally replace VH F 19 or impart FAP-specificity:

Phage display selection and Guided selection strategy with various VL as the guiding structures (improved according to McCafferty et al., 1990, Nature 348:552 and Jespers et al., 1994, Bio/Technology 12: 899) Isolation of the following human FAP-specific scFv:

scFv #13:VH #13, IgG; VL III25

scFv #46:VH #46, IgG; VL III25

scFv #50:VH #50, IgD, VL III25

Sequence of The Selected VH and VL Regions: (see Figures)

Antigen Binding Properties

ELISA: Detection of antigen specificity for human FAP

Competition for antigen binding by cF19 (detected for scFv #13)

Studies of binding to FAP⁺cells:

scFv #13 (as bivalent in minibody format) EC₅₀:8 -12 nM (see below)

scFv #50 (as bivalent in minibody format) EC₅₀:32 nM

FAP-specific immunohistological staining of tumour biopsy material (detected for scFv #13 in the minibody format)

1PCR amplification of the human VL-and VH repertoires:

a) In order to prepare the VH and VL repertoires, the various V-gene families are separately amplified from cDNA with the appropriate family-specific primers by PCR (see below).

b) All Forward/3′-primers for VH-and VL-PCR amplification are complementary to the gene sequences of the constant immunoglobulin domains (IgG, IgD, IgM, κ, λ). This allows efficient isotype-specific amplification of the V regions with very few 3′-primers. By contrast, Marks et al., 1991 (J. Mol. Biol. 222: 581) use a plurality of different 3′-primers complementary to the J-sections of the V regions.

2Preparation and cloning of a human VH repertoire:

Preparation and cloning of a human VH repertoire consisting of a large number of clones (3×10⁸) with high diversity (for method see below).

a) To ensure high diversity, commercially obtainable cDNA/RNA from different lymphoid tissues from a very great number of donors was used as the starting material for the VH repertoires in addition to freshly isolated peripheral blood lymphocytes. By using bone marrow and foetal liver, naive V repertoires should be obtained and thus the prerequisites for isolating autoantibodies are created.

Lymphoid Tissues (Number of Donors):

I) Commercial cDNA:

Peripheral blood lymphocytes, PBL (550 donors)

spleen (5 donors)

thymus gland (7 donors)

bone marrow (51 donors)

lymph nodes (59 donors)

tonsils (5 donors)

foetal livers (32 donors)

II) Commercial RNA which was subsequently circumscribed in cDNA in the laboratory (for method see Example 1, (1)(A)(2))

lymph nodes (25 donors)

III) PBL from fresh “buffy coats” (10 donors) (for method see below)

In the prior art only the following lymphoid tissues have hitherto been described as sources of V repertoires. (The combinations of the tissues and the numbers of donors are shown):

PBL (15 donors), bone marrow (4 donors), tonsils (4 donors) (Vaughan et al., 1996; Nature Biotechnology 14: 309)

spleen (3 donors) and PBL (2 donors) (Sheets et al., 1998; PNAS 95: 6157

bone marrow (Williamson et al., 1993; PNAS 90:4141)

lymph nodes (1 donors) (Clark et al., 1997; Clin. Exp. Immunol. 109: 166)

b) Moreover, the IgD repertoire was additionally amplified, as well as the IgM and IgG repertoires, to obtain a great repertoire diversity. For this, an IgD-specific PCR primer was developed (see below).

c) It proved to be very important to purify the PCR fragments of the human VH repertoire after the treatment with restriction enzymes, over an agarose gel. In subsequent cloning of this repertoire into a Phage display vector it was thus possible to achieve a very high proportion of clones with a functional scFv expression cassette. This is an essential prerequisite to obtaining a genetically stable Phage display repertoire (for method see Example 1, (1)(A)(4)).

3) Preparation of a combination repertoire consisting of a human VH repertoire and various human FAP-specific VL regions:

Definition of Combination Repertoire

Combination of a V repertoire with correspondingly complementary V-sequences by genetic engineering (complementary with regard to VH to VL and vice versa). The V-sequences used for the combination may consist of one V-sequence, a plurality of different sequences or a V repertoire.

a) Cloning strategy: In a Phage display vector the human VH repertoire was combined with a defined, non-FAP-specific VL region (dummy-VL). This dummy-VL region could very efficiently be replaced by FAP-specific VL regions using restriction cutting sites. This created the conditions for effectively combining a previously tested human VH repertoire with specific human VL, in order to guarantee a diverse combination repertoire which contains a very high proportion (greater than95%) of functional clones (in relation to the integrity of the scFv reading frame) (for method see below).

b) In order to increase the probability of selecting a fully human scFv analogous to F 19, the human VH repertoire was combined with the sequences of different human FAP-specific VL regions (VL: III10, III25, III5, III43). These human VL regions served as the guiding structures for selecting human FAP-specific VH. The FAP-specific human VL themselves had been isolated from a human VL repertoire in a previous Guided selection step with F19 VH.

c) DNA contamination of the combination repertoires with phagemid vectors which code for existing FAP-specific scFv (e.g. murine scFv from the hybridoma line F19 or the chimeric anti-FAP scFv with human VL and F19 VH), is a major technical problem. To overcome this, the following strategy proved necessary: After the Guided Selection step for the human anti-FAP VL-sequences with murine F19 VH as the guiding structure, this human VL-sequence without a VH-sequence was first sub-cloned in a plasmid (pUCBM21). Then this human VL region was excised using restriction enzymes and combined with the human VH repertoire which was already present in a Phage display vector. This prevented any FAP-specific V regions, apart from the VL-sequences of the relevant guide structure, from being introduced into the combination repertoire (for method see below).

4) Phage display selection:

The Phage display selection of the FAP-specific human V regions required the development of selective washing methods to prevent the accumulation of cross-reactive scFv (for method see below).

B) Development of Human Anti-FAP Antibodies Which Contain The Murine HCDR3 F19 (HCDR3 Retaining Guided Selection)

Method of Preparation:

1. Cloning of F19 VH

2. Preparation of human V-repertoire

Reverse transcription, PCR amplification of human VL (λ, κ) repertoires from peripheral blood lymphocytes (modified according to Persson et al. 1991, PNAS 88:2432)

Cloning of the VL repertoires in Phage display vector (pSEX81, DKFZ, Heidelberg; Breitling et al., 1991, Gene 104:147), size of repertoire: VL 10⁷ clones

Reverse transcription, PCR amplification of human VH repertoire from peripheral blood lymphocytes (improved according to Persson et al. 1991, PNAS 88: 2432), PCR amplification of the VH segment consisting of FR1+CDR1+FR2+CDR2+FR3

Cloning of a repertoire consisting of the VH segment (FR1+CDR1+FR2+CDR2+FR3) in Phage display vector (pSEX81, DKFZ, Heidelberg; Breitling et al., 1991, Gene 104:147), size of repertoire: VH 4×10⁷ clones

3. Selection of human VL regions which functionally replace VL F19:

(see (A)(3))

4. Selection of a human VH region which contains HCDR3 from F 19 and functionally replaces VH F19:

HCDR3 retaining guided selection strategy with VL III43 or VL III5 and HCDR3 F19 +human FR4 as the guiding structure

(Our own process development improved according to McCafferty et al., 1990, Nature 348:552; Jespers et al., 1994, Bio/Technology 12:899; Rader et al., 1998, PNAS 95:8910)

Isolation of the following human FAP-specific scFv, which contain murine HCDR3 F19:

scFv #34: VH #34, IgG; VL III43

scFv #18: VH #18, IgG; VL III43

Structure (see Figures)

Antigen binding properties

ELISA: detection of antigen specificity for human FAP

competition for antigen binding by cF 19 and mAb F19

Studies of binding to FAP⁺cells:

scFv #34 and #18 (monovalent) EC₅₀: about 6 nM

FAP-specific immunohistological staining of tumour biopsy material (as an scFv #34-minibody)

1.1 RNA isolation

The MRNA source used was isolated total RNA from fresh lymphocytes from a total of 10 buffy coats.

In order to isolate the lymphocytes from buffy coat, 15 ml of Ficoll (LYMPHOPREP) were placed at ambient temperature in a 50 ml Falcon Tube and covered with 30 ml of buffy coat diluted 1:4 in RPMI medium. After centrifuging for 30 min at 700 g, the interphase was removed and after the addition of 40 ml of RPMI medium, centrifuged for 5 min at 700 g. The cell pellet was then washed once more with RPMI medium and once with PBS. The cells were centrifuged after the last washing step and 200 μl of RNA-Clean™ solution (AGS, Heidelberg) were added per 106 cells. Immediately after the addition of the denaturing solution the cells were homogenised by repeatedly passing up and down through a coarse cannula (size 1) and then through a finer cannula (size 18). The thin liquid lysate was mixed with 1/10 volume chloroform (p.a.), shaken thoroughly and incubated on ice for 5 min. After centrifuging (15 min at 12000 g), the supernatant was roughly removed and mixed with an equal volume of isopropanol, incubated for 45 min at 4° C. and then centrifuged at 12000 g for 45 min. The supernatant was carefully poured off and the pellet was washed with ice-cold 70% ethanol. The RNA pellet was then washed again with components of the RNA-Quick-Prep (Pharmacia). To do this, the pellet was taken up in a mixture of 113 μl of extraction buffer, 263 μl of LiCl solution and 375 μl of Cs-trifluoroacetate, mixed thoroughly (Vortex) and centrifuged in an Eppendorf centrifuge tube (12000g). The RNA pellet was again washed with 70% ethanol, air-dried for 10 min and adjusted with H₂O to a concentration of 1μg/μl.

Alternatively, the total RNA was isolated using an RNA isolation column made by QIAGEN (Midi) according to the manufacturer′s instructions.

The mRNA was prepared from total RNA using the Oligotex-Kit (Midi) made by QIAGEN. The method used was in accordance with the manufacturer's instructions. The isolated mRNA was mixed with 1/10 volume of 2.5 M RNAse-free K-acetate, pH 5.2, and precipitated by the addition of 2.5 volumes of ethanol p.a. at −20° C. for 2 hours or overnight. After centrifuging (45 min, 13000 g, 4° C.) the mRNA was washed twice with ice-cold 70% ethanol (centrifugation for 5 min at 12000 g, 4° C.) and after brief air-drying dissolved in 10-20 μl of RNAse-free H₂0. In order to estimate the concentration, the mRNA was compared with a total RNA standard dilution series. In order to do this, 1 μl of the sample to be measured was combined with 10 μl of ethidium bromide solution (1 μg/ml), dripped onto a film and compared with the standardised concentration using a Uv lamp. The mRNA was used directly for the cDNA synthesis or frozen for storage at −80° C.

1.2 cDNA Synthesis of the Human VH Regions

IgG, IgM and IgD specific VH-cDNA was prepared with mRNA using the cDNA Synthesis Kit produced by Boehringer-Mannheim and Amersham. The first cDNA strand was synthesised with the Ig-specific primers HuIgGl-4 RT for the IgG library, HuIgM-RT for the IgM library or HulgDelta for the IgD library. Optionally, oligo(dT) and oligo-hexa-nucleotides were used. The cDNA synthesis was carried out with 100 ng of mRNA according to the manufacturer's instructions; to detach the secondary structures the MRNA had to be heated to 70 ° C. for 10 min immediately before use. The cDNA was synthesised in a 20 μl mixture with AMV-Reverse transcriptase in a Thermocycler for 60 min at 42° C. The quality of the cDNA was checked by PCR amplification using the pair of primers HuIgGFOR and HuVHB 1, by way of example. For this purpose 10ⁿ dilutions of the cDNA were used as template and the maximum dilution at which a specific band of the PCR product was still detectable in agarose gel after 36 cycles was determined.

1.3 PCR Amplification of the Human VH Repertoire

The cDNA of each human lymphatic organ was used separately as a Template for the PCR amplification of the VH regions. Six separate PCR batches were set up from each lymphatic organ, one of the six VH-specific 5′ primers (HuVHB1 to HuVHB6) being combined with one of the isotype-specific 3 ′ primers HuIgGFOR, HuIgMFOR or HuIgDFOR. The amplification was carried out in a 50μl reaction mixture with 1 μl of Template cDNA (200 pg), 25 mM MgCl₂, 5 μl of Goldstar reaction buffer, 200 μM of each dNTP (Pharmacia) and 25pmol of each primer. After 10 min at 95° C., 0.6 U of Goldstar-polymerase was added and the preparation was coated with PCR wax. Thirty-six amplification cycles were carried out, each with 15 s denaturing at 94° C., 30 s addition at 52-55° C. and 30 s elongation at 72 ° C. After the last amplification step had ended, an additional elongation was carried out for 15 min at 72° C.

In order to introduce the restriction cutting sites Nco I and Hind III onto the amplified VH regions a second PCR amplification was carried out with the primers extended by the restriction cutting sites (HuIgGFORHINDIII, HuIgMFORHINDIII, HulgDHINDIII as the 3′ primers and HuVHB INCOI to HuVHB6NCOI as the 5 ′ primers). One microlitre of the reaction solution of the first PCR mixtures was used as the template. The second PCR amplification was carried out over 15 cycles with in each case, 15 s denaturing at 94° C., 30 s addition at 65° C. and 30 s elongation at 72 ° C. The amplification step was followed once again by an additional elongation step for 5 min at 72 ° C. The amplified materials which were based on the same isotype were combined and, in order to reduce the volume, precipitated by the addition of 1/10 volume of Na-acetate, pH 5.2, and 2.5 volumes of ethanol p.a. For 2 hours at −20° C. and dissolved in TE buffer. In order to eliminate the primers, the precipitated PCR fragments were separated on a 1.5% agarose gel and the 400 Bp fragment of the VH region was excised. The fragment was isolated according to the manufacturer′s instructions using the QIA Exll-Kit made by QIAGEN (Hilden). Elution was performed with preheated elution buffer (EB) for 5 min at 50 ° C.

1.4 Digestion of the PCR-amplified VH Regions with Restriction Enzymes

The gel-purified VH regions (of the three isotypes) were first digested in a 100 μl mixture with 70 U of Hind III for 2 hours in buffer B and then incubated for a further 2 hours by the addition of 20 μl of buffer H, 60 U of NcoI and topping up to 200 μl. Any digested overhangs were eliminated using the QIA-Quick PCR-Kit and the fragments were eluted with preheated EB buffer. The eluate was purified once more over a 1% agarose gel and eluted with the QIA Exll Kit in 25 μl of EB buffer. It was found that this additional gel purification step significantly increases the percentage of functional inserts after ligation into the vector. The digested PCR fragments were divided into aliquots and stored at −20° C.

1.5 Ligation of the Human VH Repertoire into a Phagemid Vector

A Phage display vector pSEX81 which already contained the human VL-sequence of a hapten-specific Ab (Dummy VL-sequence) was used to clone the PCR-amplified VH repertoire. 20 μg of vector pSEX81(VH&VLphox) were digested in a total volume of 125 ,μl with 40 U of NcoI (Boehringer-Mannheim) and 60 μl of Hind III (Boehringer-Mannheim) in buffer H for 2 hours at 37° C. After the addition of 30 μl of 6-times concentrated Loading Buffer (30% glycerol, 30 mM EDTA) the digestion mixture was heated to 65° C for 10 min and slowly cooled at ambient temperature. Vector DNA was separated from the insert in a 1% agarose gel and isolated using the QIAGEN Gel elution kit. The elution was done twice, each time with 50 μl of elution buffer (preheated to 50° C.) For 5 min. The elution fractions were pooled and the cut vector DNA was precipitated by the addition of 1/10 volume of sodium acetate, pH 5.2, and 2.5 volumes of ethanol p.a. at −20° C. For 2 hours. If necessary the vector DNA thus cut may also be stored at −20° C. After centrifuging for 30 minutes (13000 g, 4° C. ) and washing with −20° C. cold 70% ethanol, the DNA was dried and dissolved in 50 μl of 10 mM TRIS pH 7.9.

In order to estimate the precise amount for the subsequent ligation, 2 μl of the vector DNA was compared with standardised DNA fragments (High-Mass Ladder, Gibco Life Technologies). For a direct comparison, the VH-PCR fragments prepared in Example 1, (B)(1.4) were compared with standardised DNA fragments of lower molecular weight on the same gel (Low-Mass Ladder, Gibco Life Technologies).

A ligation mixture with an equimolar insert to vector ratio proved to be ideal. In 40 μl of final volume, 500 ng of vector DNA and 50 ng Insert DNA were incubated with 1 μl of ligase and 4 μl of ligation buffer. The ligation was carried out overnight at 16° C. using the T4 DNA-ligase made by Boehringer Mannheim. The ligation reaction was stopped by the addition of 60 μl of TE buffer. The proteins were eliminated by the addition of 100 μl of chloroform/phenol mixture (1:1), brief mixing (Vortex) and subsequent centrifuging at 13000 g. The aqueous phase was removed and extracted again with chloroform to eliminate the phenol completely. 90 μl of vector DNA solution were precipitated by the addition of 9 μl of 3 M Na acetate (pH 5.2), 225 μl of ethanol p.a. and 1 μl of glycogen (Boehringer Mannheim) as carrier (see above) for 2 hours at −20° C. After centrifuging at 12000 g (4° C.) and washing with ice-cold 70% ethanol the DNA was air-dried and taken up in 25 μl of water.

Inefficient restriction digestion during the vector preparation lead to vector DNA which is uncut or cut once, with the result that in the VH repertoire cloning the size of repertoire is falsified by religation of the incompletely cut vector. For early monitoring of the completeness of the restriction digestion, the prepared vector was ligated comparatively, with and without a VH insert, transformed in E. coli and the number of clones was determined. With efficient restriction digestion of the vector, the number of clones in the vector sample without an insert was less than 1%, compared with the mixture in which the vector with a VH insert had been used.

### 2 Subcloning the human FAP-specific VL regions, combining the human VH-repertoires with various human FAP-specific VL

In order to avoid DNA contamination with existing FAP specific DNA-sequences in the construction of the scFv gene libraries, the human VL-chains selected were first cloned in the expression vector pUCBM21 (Boehringer-Mannheim). To do this, the FAP-specific VL-chains were each excised from the phagemid vector (PSEX 81), used for the selection with MluI and NotI (Boehringer-Mannheim) and recloned into the correspondingly cut pUCBM21. After transformation in E. coli a clone was picked for each VL-chain, amplified in LB_{AT}-medium and the vector DNA was isolated using the Nucleobond Kit (Macherey & Nagel). The human VL chains were excised from 15 μg of pUC-plasmid in 150 μl of restriction mixture with MluI (60U) and NotI (60U) and isolated in a 1% agarose gel. These human FAP-specific VL were cloned into correspondingly cut Phage display vectors which contain the VH repertoires. The method used to clone the VH regions was as described above. The combination banks with the different VL region were kept separate. Aliquots of these combination banks were frozen and used for the selection of fully human FAP-specific scFv.

### 3 Phage display selection

Production of the Phage-Associated scFv

In order to avoid possible growth advantages for the various VL-chains in the first round of panning, the phage-associated scFv of the various combination banks which contain the different human VL regions (see point 2) were produced independently of one another. To do this, 10 ml of 2YT_{AT} medium in a chicane shaking flask were inoculated with one aliquot of the VL/VH combination banks with an OD of 0.4 and cultivated, with agitation (180 rpm) at 37° C. until an OD of 0.8 was reached. After infection with 10¹² helper phages (New England Biolabs) incubation was carried out, without agitation, for 15 min at 37° . After subsequent incubation with agitation at 37° C. the bacteria were removed by centrifuging (4000 g for 5 min) and the pellet was resuspended in 50 ml of glucose-free 2YT_{AT} medium containing kanamycin (65 μg/ml). The phage-associated scFv was produced overnight with vigorous agitation (200 rpm) at 30° C. In order to harvest the phages the bacteria were removed by centrifuging (9000 g) and the supernatant was mixed with PEG and incubated on ice for one hour in order to precipitate it. After subsequently centrifuging for 30 minutes at 9000 g at 4° C., the precipitated phages were resuspended in 45 ml of 4° C. cold PBS and mixed with 5 ml of 5× PEG. After a further hour′s incubation on ice, the mixture was again centrifuged at 9000 g and the phage pellet was resuspended in 5 ml PBS. The phages were filtered through a 0.45 μm filter and 500 μl of each phage preparation were combined and mixed with 2 ml of 4% milk powder suspension in PBS (MPBS) for 15 min. The phage suspension was clarified by centrifuging twice with 14000 g in a bench centrifuge. The phages thus preadsorbed had to be used the same day.

Selection of Antigen-Specific scFv

Immunotubes (Nunc-Maxi-Sorb-Immunotubes 3.5 ml) immobilised with 5-30 μg CD8-FAP the day before, were used for the selection. The immobilisation was carried out at 4° C. overnight in PBS, then the tubes were washed twice with PBS and the unspecific binding sites were blocked for one hour with ROTI-Block (Roth). In order to investigate the specificity of the phage display selection, an immunotube without immobilised antigen was used for control purposes. After washing three times with PBS, the phage-associated scFv preadsorbed in MPBS were placed in the antigen-coated test tubes or the control test tubes and incubated on a roller for 2 hours.

To prepare the Plating bacteria, 20 ml of 2YTtet per mixture were inoculated with one aliquot of an XL-1-Blue overnight culture with an OD of 0.0125 and cultivated at 37° C. with agitation (180 rpm). After incubation for three hours, the Plating bacteria reached an OD of 0.8 and were then available for this time for infection with the eluted phages.

One hour before infection, the phage suspensions were emptied out of the Immunotubes. Then, the Immunotubes were washed to eliminate any unspecific and cross-reactive scFv. In the first round of panning the preparations were washed 10 times with TPBS (0.1% Tween 20) and then 10 times with PBS. The stringency was increased in the second and third rounds of panning by extending the washing steps to 15 times with TPBS (2^{nd} round of panning) and 20 times with TPBS (3rd round of panning) as well as by increasing the concentration of Tween20 to 0.5%. To increase the stringency further, in the last two rounds of panning, a vortex was briefly used during the washing with TPBS in order to mix the washing solution more thoroughly.

The final washing solution was discarded, and 1 ml of 1 M TEA (triethylamine) was added to the immunotubes. After incubation for five minutes in a roll incubator, the eluted phages were neutralised with 0.5 ml of 1 M TRIS, pH 7.4 and added directly to the 20 ml of plating bacteria for infection.

After incubation for 15 min without agitation at 37° C., the bacteria were agitated for 45 min and removed by centrifuging at 3000 g for 10 min. The bacteria were resuspended in 500 μl of 2YT medium and incubated on large SOBGAT plates (15 cm) overnight at 37° C. For harvesting, the cells were scraped from the plate with LBAT medium, mixed with 25% final concentration of glycerol and frozen in aliquots at −80° C. or used for inoculation of another round of amplification. The phage titre of each round of panning was determined by titration of 0.01-10 μl of the infected plating bacteria. In order to determine the specific concentration, in each selection round the number of eluted phages from CD8-FAP immobilised immunotubes was compared with that of the corresponding control immunotubes without an antigen. The ratio of quantities of the eluted phages from the antigen-coated immunotubes and the uncoated immunotubes yielded the concentration factor.

An increase in the concentration factor after successive amplification round indicated a concentration of specifically binding phages.

### EXAMPLE 2

Expression of the Human FAP-Specific scFv Derivatives

Screening process on a microtitre scale for evaluating phage display-selected scFv

The scFv-pIII-fusion proteins expressed using pSEX81 may be used both for Screening, i.e., sampling, and for analysis of scFv clones selected from phage display banks.

Bacterial Production of scFv-pIII-Fusion Protein on a Microtitre Scale

300 μl aliquots of 2YT_{GAT} were inoculated with colonies set out individually on LB_{GAT} plates and incubated overnight (o-n) in 96-well microtitre plates (Beckman) at 37° C. and 300 rpm with agitation. If the colonies to be analysed were not to be stored frozen, this initial incubation was carried out in U-shaped 96-well tissue culture plates (Greiner). The next morning, 10 μl aliquots of these o-n cultures were transferred into a fresh 100 μl of 2YT and incubated again, with agitation, in U-shaped 96-well tissue culture plates in a damp chamber at 37° C. The residue of the cultures left in the Beckman microtitre plates was able to be mixed with glycerol at 20% and frozen at −80° C. The growth of the 100 μl of cultures could be checked if necessary with an ELISA Reader at a filter wavelength of 630 nm. After about 6-8 h, the cultures were centrifuged at 1200 rpm (5 min, RT) and the supernatants were removed with a multichannel pipette. The pelleted bacteria were resuspended in 100 μl aliquots of 2YT_{AT} (without glucose) including 50 μM IPTG and incubated o-n with agitation in the damp chamber at 30° C. and 300 rpm. After o-n incubation the cultures were each mixed with 25 μl of 0.5% Tween and incubated with agitation for a further 3-4 h to achieve partial lysis. Finally, the cultures were centrifuged for 10 min at 1200 rpm and the supernatants were carefully removed. These were used directly for Western blot analysis or after preadsorption used in the ELISA.

Production of scFv-pIII-Fusion Protein on the ml Scale

If only small numbers of clones were to be investigated for their expression and/or for the functionality of the scFv-pIII-fusion protein expressed, the overnight precultivation as well as the main cultivation of the bacteria were carried out in a volume of 3-10 ml in test tubes or in 50 ml PP-test tubes with agitation at about 200 rpm. If the bacterial growth had reached its logarithmic phase (OD₆₀₀ₙₘ about 0.7), the cultures were centrifuged (2500 rpm, 5 min, room temperature (RT)) and resuspended in an equal volume of fresh SB_{AT} or 2YT_{AT} including 50 μM-IPTG for induction. After o-n incubation at 25-30° C. either the cultures were mixed with Tween 20 (ad 0.1%) and the supernatants were removed after 3 h of further incubation. However, in order to increase the concentration of the fusion proteins, the bacterial pellet could also be opened up (see below).

The scFv-9gIII-fusion proteins were used to demonstrate the integrity of the reading frames of the scFv-coding region (Western blot) and to investigate the FAP specificity of the scFv selected in the ELISA on immobilised FAP or in the cell analyser on FAP+ cells. An anti-giIII-specific monoclonal antibody combined with a peroxidase-or alkaline phosphatase-conjugated detection antibody (Western-Blot and ELISA) was used to detect the scFv-gIII-fusion proteins. In the case of cell binding studies with the scFv-gIII proteins in the cell analyser, an FITC-labelled detection antibody was used.

Prokaryotic Expression

Media

All the data relate to a final volume of 1 L, the pH was adjusted to 7.0. The following additions of media were filtered sterile and optionally added to the autoclaved medium. G: 100 mM glucose (stock solution: 2 M), A: ampicillin 100 μg/ml, T: tetracycline 12.5 μg/ml, K: kanamycin 50 μg/ml

Liquid Media for the Bacterial Culture:

| | | | |
|---|---|---|---|
| BHI | Brain Heart Infusion (DIFCO) | 35 | g |
| | yeast extract | 5 | g |
| dYT | peptone | 17 | g |
| | yeast extract | 10 | g |
| | NaCl | 5 | g |
| LB | peptone | 10 | g |
| | yeast extract | 10 | g |
| | NaCl | 5 | g |
| SB | peptone | 30 | g |
| | yeast extract | 10 | g |
| | MOPS | 10 | g |
| SOC | peptone | 20 | g |
| | yeast extract | 5 | g |
| | NaCl | 10 | mM |
| | KCl | 2.5 | mM |

After autoclaving, sterile MgCl₂ and MgSO₄ are added ad 10 mM in each case, as well as sterile glucose ad 20 mM

Agar dishes

| BHI | (amounts per Petri dish) | | |
|---|---|---|---|
| | BHI (without yeast) | 30 | ml |
| | agar agar | 1% | |
| | saccharose (60%) | 0.5 | ml |
| | horse serum | 2.5 | ml |
| | yeast extract (20%) | 1 | ml |
| | glucose (20%) | 0.5 | ml |

saccharose, serum, yeast extract, glucose are all added sterile

| LB | LB medium +1.5% (w/v) agar agar | | |
|---|---|---|---|
| SOB | peptone | 20 | g |
| | yeast extract | 5 | g |
| | NaCl | 0,5 | g |
| | agar agar | 15 | g |

After autoclaving, sterile MgCl₂ is added ad 10 mM

Other abbreviations: G: glucose, A: ampicillin, T: tetracycline, K: kanamycin

Bacterial Expression of scFv in E. coli

pOPE vectors and derivatives obtained therefrom were used to prepare a simple soluble scFv derivative with cmyc-and HIS₆-Tag in E. coli (Dubel et al., 1993; Gene 128: 97-101). The scFv expression in E. coli and the purification thereof are carried out according to the processes of Moosmayer et al., 1995 (Ther. Immunol. 2: 31-40).

The scFv was produced in E.coli XL1-Blue in volumes of 3-100 ml. The incubation took place either in test tubes or in 50 ml PP-test tubes with agitation at about 200 rpm or in Erlenmeyer chicane flasks at 180 rpm in LB or 2YT medium. The media were buffered with 1/10 volume MOPS (pH 7) and mixed with tetracycline (12.5 μg/ml) for the strain XL1-Blue.

2YT_{GAT} or LB_{GAT} was inoculated with colonies separated out on LB_{GAT} plates to form a preliminary culture and incubated o-n at 37° C. with agitation. The next day the main culture was inoculated 1:50 therewith and incubated at 37° C. For induction, the centrifuged bacteria (2500 rpm, 1000×g, 10 min, RT) were taken up in an equal volume of medium (without glucose) with 50 μM-IPTG and agitated for 2-3 h at 22-25° C. and 220 rpm. The bacterial pellet was harvested after centrifugation at 1000×g (10 min, RT) and broken up as follows. The harvested pellets of the induced E.coli cultures were taken up in 1/20-1/30 volume of ice-cold PBS and thoroughly resuspended, incubated for about 30 min on ice with occasional mixing and flash-frozen in liquid nitrogen or in a mixture of ethanol and dry ice. The frozen sample could then be stored at −80° C. To break it up, the sample was slowly thawed and subjected to ultrasound treatment (25-30 cycles while cooling with ice water) until it was homogeneous and clear. In order to obtain the entire soluble fraction of bacterial protein, the sample was centrifuged for 20 min at 13000 rpm, the supernatant was carefully removed and the pellet was discarded. For longer storage, if desired, the supernatants were mixed with BSA (ad 1%), flash frozen and stored at −80° C.

In the preparation of scFv F19 in E. coli, a drastic deterioration in the functionality of the recombinant proteins was observed if excessively rich (SB medium) or unbuffered culture media were used.

Expression of scFv Derivatives in Proteus Mirabilis L VI

Monomeric scFv as well as dimeric scFv (minibodies) were expressed in Proteus mirabilis. The expression and purification process was analogous to that which we have already published for soluble monovalent scFv (Rippmann et al., 1998, Applied and Environmental Microbiology 64: 4862-4869).

Transformation of Plasmid DNA in P. mirabilis LVI

The incubation of P. mirabilis L VI was carried out in Erlenmeyer flasks (without chicanes) at greater than 200 rpm. For transformation of the L VI bacteria they had to be in the stationary growth phase (OD₅₅₀ about 6). To do this, 20 ml of a BHI_{K} culture were inoculated 1:20 from a 4° C. culture and incubated o-n at 37° C. with agitation. Every 100 μl of the o-n culture were mixed with 20 μl of the prepared plasmid and 150 μl of PEG (including 0.4 M-saccharose) and stored on ice for 10 min. The temperature shock lasted for 5 min with occasional gentle agitation in a water bath at 37° C. The transformed LVI-bacteria were taken up in 1 ml of BYS medium (1 ml BHI, 0.5% yeast extract, 1% saccbarose) and incubated for 3 h with vigorous agitation in a small steep-walled container at 37° C. One hundred microlitres of each transformation mixture were plated out on a BHIₖ plate. After 24-48 h incubation (37° C.) significantly large colonies were pricked out using a sterile spatula and transferred into 20 ml of BHIₖ medium. After o-n growth and microscopic monitoring for the presence of L-form bacteria, this culture was mixed with cryomedium and frozen at −80° C. Unfrozen transformed P. mirabilis cultures remained viable for at least 4 weeks when stored at 4° C. In order to induce expression in transformed P. mirabilis, two successive o-n or 11 -12 h preliminary cultures were inoculated (20 ml each) and incubated at 30° C., the first of them from a 4° C. culture. Depending on the density of the preliminary culture achieved and the length of incubation of the following culture, it was always overinoculated 1:10 or 1:20. The BHIₖ induction cultures (including 0.5 mM-IPTG) had a volume of 20-50 ml and were also inoculated, then incubated at 30° C. with agitation for at least 11 h. Before the harvesting of the bacteria, the OD₅₅₀ (≧4), the pH (7.5-8.5) and the optical appearance of the L forms were examined under the microscope. The expression culture was centrifuged (5000 rpm, 3800×g, 4° C.) and the pellet was discarded. The supernatant could be used directly for ELISA or Western Blot analysis or it could be purified.

In this study, the minibodies were purified by IMAC (immobilized metal affinity chromatography). One mililtre HiTrap columns made by Pharmacia Biotech were used for this. Gel chromatography was carried out as the second purification step.

Before the induction supernatant was applied, it was thoroughly dialysed against 5 L of cold PBS (pH 8), then ultracentrifuged for at least 30 min (113000× g, 4° C., rotor: Beckman 45 Ti). The column had to be charged with Zn²⁻ ions before each purification. The solutions used were filtered sterile beforehand to prevent clogging by the particles. Residues of metal ions were eliminated with 5 ml of 50 mM EDTA. After rinsing with 10 ml of H₂0_{bid} charging was carried out with 10 ml of 100 mM ZnSO₄. After rinsing again with 20 ml of H₂0_{bid} the column was equilibrated with 10 ml of PBS (pH 8). The supernatant was applied to the column using a peristaltic pump (1.5 ml/min), followed by a washing step (10 ml PBS including 5-20 mM imidazole). Elution was carried out in 1 ml fractions with 10 ml PBS including 300 mM imidazole. The elution fractions were stored on ice.

For the gel chromatography, a Superdex 200 column (10/30) made by Pharmacia Biotech was used in conjunction with an FPLC apparatus made by the same manufacturer. The IMAC-purified sample was centrifuged for 5 min (13000 rpm, 4° C.) before the injection.

After the equilibration of the pump system and column with the chosen elution buffer (PBS, pH 8), 500 μl (corresponding to 0.75-1 mg) of IMAC-purified MB #34 were injected into the system, pumped at a flow rate of 0.5 ml/min, detected with a UV-detector and automatically collected in 500 μl fractions.

Structure of the Recombinant Human Antibodies

The prokaryotic and eukaryotic expression of the human recombinant anti-FAP-antibodies took place as monovalent scFv and bivalent scFv (so-called minibodies). The structure of the minibodies produced and the expression cassettes used for this purpose is comparable with those described by Hu et al. 1996 (Cancer Res. 56: 3055-61). In addition, these minibodies have a c-myc domain at the C-terminus for immunological detection (with the monoclonal antibody 9E10) and a HIS₆ domain for chromatographic purification. The cmyc- and HIS₆-coding sequences correspond to those from pOPE 101 (S. Dübel, University of Heidelberg).

Structure of the minibodies:

N-signal sequence-scFv(VH-linker-VL)-hinge-linker-CH3-cmyc-HIS₆-C

Prokaryotic Expression of Antibody Proteins According to the Invention

The expression vectors used and the processes for the expression and purification of monovalent scFv derivatives in E. coli (Moosmayer et al., 1995, Ther. Immunol. 2: 31-40) and Proteus mirabilis LVI (Rippmann et al., 1998, Applied and Environmental Microbiology 64: 4862-4869) are known from the prior art. The vector pACK02scKan and the processes from Rippmann et al., 1998 were also used to prepare and purify a minibody in Proteus mirabilis L VI.

Eukaryotic Expression of the Antibody Proteins According to the Invention

The minibodies described were also prepared in mammalian cells. The expression vectors used for the minibody expression cassettes were: pAD-CMV-1 and a pg1d105 derivative.

Transient Expression in COS Cells

For transfecting COS 7 cells, the expression vector was first amplified in E. coli (XL1-Blue) and then purified. The vector DNA was adjusted to a concentration of 1 μg/μl under sterile conditions and stored at −20° C.

On the day before the transfection, 5×10⁵ COS7 cells were seeded in a cell culture Petri dish (8 cm diameter, Greiner ) in DEMEM 10%FCS and incubated for 16 h at 37° C. in a CO₂ heating cupboard. On the day of the transfection, a suspension was prepared consisting, per Petri dish, of 1 ml of OptiMEM (Gibco), 35 ,μl of lipofectamine (Gibco Life Science) and 10 μg of expression vector DNA. After incubation at ambient temperature for 45 min, a further 4 ml of OptMEM were added and the suspension was carefully pipetted over the cells which had previously been washed with PBS. The solution was distributed by gentle tilting and incubated for 5 hours at 37° C. The Petri dish was filled with 5 ml of preheated DEMEM 20% FCS and incubated for 16 h at 37° C. Then, the incubation medium was carefully suction filtered and replaced by 10 ml of OptiMEM. After another incubation for 48 hours at 37° C., the supernatant was removed for harvesting and the cells were removed by centrifuging at 700 g. A further centrifugation step at 12000 g pelleted the remaining cell fragments. The supernatant was either ultracentrifuged for 30 min (60000×g for 30 min) and then added to an IMAC column (Amersham-Pharmacia) or evaporated down to 1/40 to 1/80 volume in centrifugal concentrators with a 30 kDa separation threshold (Fugisept-Midi or MaxiRöhrchen, Intersept). The centrifugation was carried out according to the manufacturer's instructions at 6000 g for about 6 hours. The concentrated protein solution was mixed with 1% BSA, divided into 100 μl aliquots and after flash freezing in N₂ stored at −80° C.

Stable Expression in CHO Cells:

Stable transfectants of CHO DG44 were prepared for the expression of FAP-specific minibodies.

Transfection:

1st day: 2×10⁵ cells were seeded in one well of a 6-well plate

2nd day: Careful suction filtering of the cell culture supernatant and subsequent addition of 800 μl CHO-SFM II medium plus HT supplement (Gibco BRL).

Preparation of the transfection suspension: 6 μl of lipofectamine+200 μl of CHO-SFM II with HT supplement+3 μl (3 μg) of expression vector. The suspension was mixed and carefully added to the cells.

3rd day: Change of medium: addition of CHO-SFM II without an HT supplement.

The change of medium was repeated regularly. For the gene amplification and for increasing the expression of foreign genes, methotre×ate was added to the medium from a period 10-14 days after the transfection. The methotrexate concentration was slowly increased; the concentrations were between 10 and 1000 nM.

The minibodies were produced in T-culture flasks or in a bioreactor.

Determining the Apparent Cell Binding Affinity of the Recombinant Anti-FAP Antibodies

FAP+ cells were incubated in parallel batches with various concentrations of mono-or bivalent scFv derivatives. The binding of these recombinant antibodies was determined using an FITC-labelled detection antibody in a cell analyser (Coulter). The concentration of the scFv derivatives at which half the maximum saturation of the binding signal was achieved was chosen as a measurement of the apparent affinity.

### EXAMPLE 3

### Sequence

The sequences are shown here by way of example. Smaller mutations, e.g. the substitution of one or a few amino acids or the nucleotides coding therefor are also encompassed by the invention.

VH13 Protein sequence such as may be found in the minibody vector, for example. The first amino acid may also be an E (glutamate).

| | |
|---|---|
| QVQLVESGGTLVQPGGSLRLSCAASGFTFSSYAMSWIRQAPGKGLEWVSGISASGGYIDYA | (SEQ ID NO:1) |
| DSVKGRVTISRDNSKNMAY | |
| LQMSSLRAEDTALYYCAKGGNYQMLLDHWGQGTLVTVSSASTKGPKL | |

Nucleotide sequence corresponding to VH13

| | |
|---|---|
| CAGGTACAGCTGGTGGAGTCTGGGGGAACCTTGGTACAGCCTGGGGGGTCCCTGAGACT | (SEQ ID NO:5) |
| CTCCTGTGCAGCCTCTGGATT | |
| CACCTTTAGCAGCTATGCCATGAGCTGGATCCGCCAGGCTCCAGGGAAGGGGCTGGAGT | |
| GGGTCTCAGGTATTAGTGCTA | |
| GTGGTGGTTATATAGACTATGCCGATTCCGTGAAGGGCCGGGTCACCATCTCCAGAGAC | |
| AATTCCAAGAACATGGCATAT | |
| CTACAAATGAGCAGCCTGAGAGCCGAGGACACGGCCCTTTATTACTGTGCGAAAGGAGG | |
| CAACTACCAGATGCTATTGGA | |
| CCACTGGGGCCAGGGAACCCTGGTCACCGTCTCCTCAGCCTCCACCAAGGGCCCAAAGC | |
| TT | |

VH 46 Protein sequence.

| | |
|---|---|
| QVQLVQSGAEVKKDGASVKVSCKATGGTFSGHAISWVRQAPGQRLEWMGEISPMFGTPNY | (SEQ ID NO:2) |
| AQSFQGRVTITADESTSYME | |
| VSSLRSEDTATYYCARGANYRALLDYWGQGTLVTVSSASTKGPKL | |

Nucleotide sequence corresponding to VH46 such as may occur in the minibody, for example. The sixth nucleotide may also be an A instead of a G—a silent mutation, hence having no effect on the amino acid sequence.

| | |
|---|---|
| CAGGTACAGCTGGTGCAGTCTGGGGCTGAAGTGAAGAAGGATGGGGCCTCAGTGAAGG | (SEQ ID NO:6) |
| TCTCCTGCAAGGCTACTGGAGG | |
| CACTTTCAGCGGTCACGCTATCAGTTGGGTGCGACAGGCCCCTGGGCAAAGACTTGAGT | |
| GGATGGGGGAGATCAGCCCTA | |
| TGTTTGGAACACCAAACTACGCACAGAGCTTCCAGGGCAGAGTCACGATTACCGCGGAC | |
| GAATCTACGAGTTACATGGAG | |
| GTGAGCAGCCTGAGATCTGAGGACACGGCCACTTATTACTGTGCGAGAGGTGCGAACTA | |
| CCGGGCCCTCCTTGATTACTG | |
| GGGCCAGGGAACCCTGGTCACCGTCTCCTCAGCCTCCACCAAGGGCCCAAAGCTT | |

VH50 Protein sequence as occurs in the minibody. Again, the same applies as for VH13: The first amino acid may also be an E.

| | |
|---|---|
| QVQLVESGGGLVQPGGSLRLSCAASGFTFSNYWMSWVRQAPGKGLEWVANIKQDGSEKY | (SEQ ID NO:3) |
| YVDSVKGRFTISRDNAKNSLY | |
| LQMNSLRAEDTAVYYCARGSLCTDGSCPTIGPGPNWGQGTLVTVSSAPTKAPKL | |

Nucleotide sequence corresponding to VH50 as occurs in the minibody, for example

| | |
|---|---|
| CAGGTACAGCTGGTGGAGTCTGGGGGAGGCTTGGTCCAGCCTGGGGGGTCCCTGAGACT | (SEQ ID NO:7) |
| CTCCTGTGCAGCCTCTGGATT | |
| CACCTTTAGTAACTATTGGATGAGCTGGGTCCGCCAGGCTCCAGGGAAGGGGCTGGAGT | |
| GGGTGGCCAACATAAAGCAAG | |
| ATGGAAGTGAGAAATACTATGTGGACTCTGTGAAGGGCCGATTCACCATCTCCAGAGAC | |
| AACGCCAAGAACTCACTGTAT | |
| CTGCAAATGAACAGCCTGAGAGCCGAGGACACGGCTGTGTATTACTGTGCGAGAGGTTC | |
| ACTCTGTACTGATGGTAGCTG | |
| CCCCACCATAGGGCCTGGGCCAAACTGGGGCCAGGGAACCCTGGTCACCGTCTCCTCAG | |
| CACCCACCAAGGCTCCGAAGC | |
| TT | |

VLIII25 Protein

| | |
|---|---|
| DIQMTQSPSSLSASTGDRVTITCRASQDISSYLAWYQQAPGKAPHLLMSGATTLQTGVPSRFS | (SEQ ID NO:4) |
| GSGSGTDFTLTITSLQS | |
| EDFATYYCQQYYIYPPTEGQGTRVEIKRTVAAPSVFAA | |

Nucleotide sequence corresponding to VLIII25

| | |
|---|---|
| GACATCCAGATGACCCAGTCTCCATCCTCACTCTCTGCATCTACAGGAGACAGAGTCAC | (SEQ ID NO:8) |
| CATCACTTGTCGGGCGAGTCA | |
| AGATATTAGCAGTTATTTAGCCTGGTATCAACAGGCACCCGGGAAAGCCCCTCATCTCCT | |
| GATGTCTGGAGCAACCACTT | |
| TACAGACTGGAGTCCCATCAAGGTTCAGCGGCAGTGGATCTGGGACAGATTTCACTCTC | |
| ACCATCACGTCCCTGCAGTCT | |
| GAAGATTTTGCAACTTATTACTGTCAACAGTATTATATTTACCCTCCGACGTTCGGCCAA | |
| GGGACCAGGGTGGAAATCAA | |
| ACGAACTGTGGCTGCACCATCTGTCTTCGCGGCCGC | |

Protein VH34 with the first 8 amino acids of CH:

| | |
|---|---|
| QVQLQQSGAEVKKPGSSVKVSCKASGGTFSTHTINWVRQAPGQGLEWMGGIAPMFGTANY | (SEQ ID NO:9) |
| AQKFQGRVTITADKSTSTAY | |
| MEMSSLRSDDTAVYYCARRRIAYGYDEGHAMDYWGQGTLVTVSSASTKGPKL | |

Nucleic acid sequence corresponding to VH34:

| | |
|---|---|
| CAGGTACAGCTGCAGCAGTCAGGGGCTGAGGTGAAGAAGCCTGGGTCCTCGGTGAAGG | (SEQ ID NO:12) |
| TCTCCTGCAAGGCTTCTGGAGG | |
| CACCTTCAGCACCCATACTATCAACTGGGTGCGACAGGCCCCTGGACAAGGGCTTGAGT | |
| GGATGGGAGGGATCGCCCCTA | |
| TGTTTGGTACAGCAAACTACGCACAGAAGTTCCAGGGCAGAGTCACAATTACCGCGGAC | |
| AAATCCACGAGCACAGCCTAC | |
| ATGGAGATGAGCAGCCTGAGATCTGACGACACGGCTGTGTATTACTGTGCAAGAAGAAG | |
| AATCGCGTACGGTTACGACGA | |
| GGGCCATGCTATGGACTACTGGGGTCAAGGAACCCTTGTCACCGTCTCCTCAGCCTCCAC | |
| CAAGGGGCCAAAGCTT | |

VH18 with some amino acids of CH1:

| | |
|---|---|
| QVQLVQSGAELKKPGSSMKVSCKASGDTFSTYSINWVRQAPGQGLEWMGWNPSGGSTSY | (SEQ ID NO:10) |
| AQKFQGRVTMTRDTSTSTVY | |
| MELSSLRSEDTAVYYCARRRIAYGYDEGHAMDYWGQGTLVTVSSASTKGPKL | |

Nucleic acid sequence corresponding to VH 18:

| | |
|---|---|
| CAGGTGCAGCTGGTGCAGTCTGGGGCTGAGTTGAAGAAGCCTGGGTCCTCGATGAAGGT | (SEQ ID NO:13) |
| CTCCTGCAAGGCTTCTGGAGA | |
| CACCTTCAGCACCTATTCTATCAACTGGGTGCGACAGGCCCCTGGACAAGGGCTTGAGT | |
| GGATGGGAGTAATCAACCCTA | |
| GTGGTGGTAGCACAAGCTACGCACAGAAGTTCCAGGGCAGAGTCACCATGACCAGGGA | |
| CACGTCCACGAGCACAGTTTAC | |
| ATGGAGCTGAGCAGCCTGAGATCTGAAGACACGGCCGTGTATTACTGTGCGAGAAGAA | |
| GAATCGCGTACGGTTACGACGA | |
| GGGCCATGCTATGGACTACTGGGGTCAAGGAACCCTTGTCACCGTCTCCTCAGCCTCCAC | |
| CAAGGGCCCAAAGCTT | |

VL chain 11143

| | |
|---|---|
| DIQMTQSPSSLSASTGDRVTITCRASQDISSYLAWYQQAPGKAPHLLMSGATTLQTGVPSRFS | (SEQ ID NO:11) |
| GSGSGTDFTLTISSLQA | |
| EDVAVYYCQQYYRTPFTFGQGTKLEIKRTVAAPSVFAA | |

Nucleic acid sequence corresponding to III43:

| | |
|---|---|
| GACATCCAGATGACCCAGTCTCCATCCTCACTCTCTGCATCTACAGGAGACAGAGTCAC | (SEQ ID NO:14) |
| CATCACTTGTCGGGCGAGTCA | |
| AGATATTAGCAGTTATTTAGCCTGGTATCAACAGGCACCCGGGAAAGCCCCTCATCTCCT | |
| GATGTCTGGAGCAACCACTT | |
| TACAGACTGGAGTCCCATCAAGGTTCAGCGGCAGTGGATCTGGGACAGATTTCACTCTC | |
| ACCATCAGCAGCCTGCAGGCT | |
| GAAGATGTGGCAGTTTATTACTGTCAGCAATATTATCGTACTCCGTTTACTTTTGGCCAG | |
| GGGACCAAGTTGGAGATCAA | |
| ACGAACTGTGGCTGCACCATCTGTCTTCGCGGCCGC | |

VH 13 YOL VL III25 Protein sequence of the total antibody protein, as occurs in the minibody

| | |
|---|---|
| QVQLVESGGTLVQPGGSLRLSCAASGFTFSSYAMSWIRQAPGKGLEWVSGISASGGYIDYA | (SEQ ID NO:15) |
| DSVKGRVTISRDNSKNMAY | |
| LQMSSLRAEDTALYYCAKGGNYQMLLDHWGQGTLVTVSSASTKGPKLEEGEFSEARVDIQ | |
| MTQSPSSLSASTGDRVTITC | |
| RASQDISSYLAWYQQAPGKAPHLLMSGATTLQTGVPSRFSGSGSGTDFTLTITSLQSEDFATY | |
| YCQQYYIYPPTFGQGTR | |
| VEIKRTVAAPSVFAA | |

Nucleotide sequence corresponding to VH 13 YOL VL III25

| | |
|---|---|
| CAGGTACAGCTGGTGGAGTCTGGGGGAACCTTGGTACAGCCTGGGGGGTCCCTGAGACT | (SEQ ID NO:20) |
| CTCCTGTGCAGCCTCTGGATT | |
| CACCTTTAGCAGCTATGCCATGAGCTGGATCCGCCAGGCTCCAGGGAAGGGGCTGGAGT | |
| GGGTCTCAGGTATTAGTGCTA | |
| GTGGTGGTTATATAGACTATGCCGATTCCGTGAAGGGCCGGGTCACCATCTCCAGAGAC | |
| AATTCCAAGAACATGGCATAT | |
| CTACAAATGAGCAGCCTGAGAGCCGAGGACACGGCCCTTTATTACTGTGCGAAAGGAGG | |
| CAACTACCAGATGCTATTGGA | |
| CCACTGGGGCCAGGGAACCCTGGTCACCGTCTCCTCAGCCTCCACCAAGGGCCCAAAGC | |
| TTGAAGAAGGTGAATTTTCAG | |
| AAGCACGCGTAGACATCCAGATGACCCAGTCTCCATCCTCACTCTCTGCATCTACAGGA | |
| GACAGAGTCACCATCACTTGT | |
| CGGGCGAGTCAAGATATTAGCAGTTATTTAGCCTGGTATCAACAGGCACCCGGGAAAGC | |
| CCCTCATCTCCTGATGTCTGG | |
| AGCAACCACTTTACAGACTGGAGTCCCATCAAGGTTCAGCGGCAGTGGATCTGGGACAG | |
| ATTTCACTCTCACCATCACGT | |
| CCCTGCAGTCTGAAGATTTTGCAACTTATTACTGTCAACAGTATTATATTTACCCTCCGA | |
| CGTTCGGCCAAGGGACCAGG | |
| GTGGAAATCAAACGAACTGTGGCTGCACCATCTGTCTTCGCGGCCGC | |

VH46 YOL VL 11125 Protein sequence of the total antibody protein as occurs in the minibody, for example

| | |
|---|---|
| QVQLVQSGAEVKKDGASVKVSCKATGGTFSGHAISWVRQAPGQRLEWMGEISPMFGTPNY | (SEQ ID NO:18) |
| AQSFQGRVTITADESTSYME | |
| VSSLRSEDTATYYCARGANYRALLDYWGQGTLVTVSSASTKGPKLEEGEFSEARVDIQMTQ | |
| SPSSLSASTGDRVTITCRA | |
| SQDISSYLAWYQQAPGKAPHLLMSGATTLQTGVPSRFSGSGSGTDFTLTITSLQSEDFATYYC | |
| QQYYIYPPTFGQGTRVE | |
| IKRTVAAPSVFAA | |

Nucleotide sequence corresponding to VH46 YOL VL III25

| | |
|---|---|
| CAGGTACAGCTGGTGCAGTCTGGGGCTGAAGTGAAGAAGGATGGGGCCTCAGTGAAGG | (SEQ ID NO:23) |
| TCTCCTGCAAGGCTACTGGAGG | |
| CACTTTCAGCGGTCACGCTATCAGTTGGGTGCGACAGGCCCCTGGGCAAAGACTTGAGT | |
| GGATGGGGGAGATCAGCCCTA | |
| TGTTTGGAACACCAAACTACGCACAGAGCTTCCAGGGCAGAGTCACGATTACCGCGGAC | |
| GAATCTACGAGTTACATGGAG | |
| GTGAGCAGCCTGAGATCTGAGGACACGGCCACTTATTACTGTGCGAGAGGTGCGAACTA | |
| CCGGGCCCTCCTTGATTACTG | |
| GGGCCAGGGAACCCTGGTCACCGTCTCCTCAGCCTCCACCAAGGGCCCAAAGCTTGAAG | |
| AAGGTGAATTTTCAGAAGCAC | |
| GCGTAGACATCCAGATGACCCAGTCTCCATCCTCACTCTCTGCATCTACAGGAGACAGA | |
| GTCACCATCACTTGTCGGGCG | |
| AGTCAAGATATTAGCAGTTATTTAGCCTGGTATCAACAGGCACCCGGGAAAGCCCCTCA | |
| TCTCCTGATGTCTGGAGCAAC | |
| CACTTTACAGACTGGAGTCCCATCAAGGTTCAGCGGCAGTGGATCTGGGACAGATTTCA | |
| CTCTCACCATCACGTCCCTGC | |
| AGTCTGAAGATTTTGCAACTTATTACTGTCAACAGTATTATATTTACCCTCCGACGTTCG | |
| GCCAAGGGACCAGGGTGGAA | |
| ATCAAACGAACTGTGGCTGCACCATCTGTCTTCGCGGCCGC | |

VH 50 YOL VL III25 Protein sequence of the total antibody protein as occurs in the minibody, for example (for possible variation see VH50, above)

| | |
|---|---|
| QVQLVESGGGLVQPGGSLRLSCAASGFTFSNYWMSWVRQAPGKGLEWVANIKQDGSEKY | (SEQ ID NO:19) |
| YVDSVKGRFTISRDNAKNSLY | |
| LQMNSLRAIEDTAVYYCARGSLCTDGSCPTIGPGPNWGQGTLVTVS SAPTKAPKLEEGEFSE | |
| ARVDIQMTQSPSSLSASTG | |
| DRVTITCRASQDIS SYLAWYQQAPGKAPHLLMSGATTLQTGVPSRFSGSGSGTDFTLTITSLQ | |
| SEDFATYYCQQYYIYPP | |
| TFGQGTRVEIKRTVAAPSVFAA | |

Nucleotide sequence corresponding to VH 50 YOL VL III25

| | |
|---|---|
| CAGGTACAGCTGGTGGAGTCTGGGGGAGGCTTGGTCCAGCCTGGGGGGTCCCTGAGACT | (SEQ ID NO:24) |
| CTCCTGTGCAGCCTCTGGATT | |
| CACCTTTAGTAACTATTGGATGAGCTGGGTCCGCCAGGCTCCAGGGAAGGGGCTGGAGT | |
| GGGTGGCCAACATAAAGCAAG | |
| ATGGAAGTGAGAAATACTATGTGGACTCTGTGAAGGGCCGATTCACCATCTCCAGAGAC | |
| AACGCCAAGAACTCACTGTAT | |
| CTGCAAATGAACAGCCTGAGAGCCGAGGACACGGCTGTGTATTACTGTGCGAGAGGTTC | |
| ACTCTGTACTGATGGTAGCTG | |
| CCCCACCATAGGGCCTGGGCCAAACTGGGGCCAGGGAACCCTGGTCACCGTCTCCTCAG | |
| CACCCACCAAGGCTCCGAAGC | |
| TTGAAGAAGGTGAATTTTCAGAAGCACGCGTAGACATCCAGATGACCCAGTCTCCATCC | |
| TCACTCTCTGCATCTACAGGA | |
| GACAGAGTCACCATCACTTGTCGGGCGAGTCAAGATATTAGCAGTTATTTAGCCTGGTA | |
| TCAACAGGCACCCGGGAAAGC | |
| CCCTCATCTCCTGATGTCTGGAGCAACCACTTTACAGACTGGAGTCCCATCAAGGTTCAG | |
| CGGCAGTGGATCTGGGACAG | |
| ATTTCACTCTCACCATCACGTCCCTGCAGTCTGAAGATTTTGCAACTTATTACTGTCAAC | |
| AGTATTATATTTACCCTCCG | |
| ACGTTCGGCCAAGGGACCAGGGTGGAAATCAAACGAACTGTGGCTGCACCATCTGTCTT | |
| CGCGGCCGC | |

VH34YOL III43 Protein sequence of the total antibody protein:

| | |
|---|---|
| QVQLQQSGAEVKKPGSSVKVSCKASGGTFSTHTINWVRQAPGQGLEWMGGIAPMFGTANY | (SEQ ID NO:17) |
| AQKFQGRVTLTADKSTSTAY | |
| MEMSSLRSDDTAVYYCARRRIAYGYDEGHAMDYWGQGTLVTVSSASTKGPKLEEGEFSEA | |
| RVDLQMTQSPSSLSASTGDR | |
| VTITCRASQDISSYLAWYQQAPGKAPHLLMSGATTLQTGVPSRFSGSGSGTDFTLTISSLQAE | |
| DVAVYYCQQYYRTPFTF | |
| GQGTKLEIKRTVAAPSVFAA | |

Nucleotide sequence corresponding to VH34YOL III43:

| | |
|---|---|
| CAGGTACAGCTGCAGCAGTCAGGGGCTGAGGTGAAGAAGCCTGGGTCCTCGGTGAAGG | (SEQ ID NO:22) |
| TCTCCTGCAAGGCTTCTGGAGG | |
| CACCTTCAGCACCCATACTATCAACTGGGTGCGACAGGCCCCTGGACAAGGGCTTGAGT | |
| GGATGGGAGGGATCGCCCCTA | |
| TGTTTGGTACAGCAAACTACGCACAGAAGTTCCAGGGCAGAGTCACAATTACCGCGGAC | |
| AAATCCACGAGCACAGCCTAC | |
| ATGGAGATGAGCAGCCTGAGATCTGACGACACGGCTGTGTATTACTGTGCAAGAAGAAG | |
| AATCGCGTACGGTTACGACGA | |
| GGGCCATGCTATGGACTACTGGGGTCAAGGAACCCTTGTCACCGTCTCCTCAGCCTCCAC | |
| CAAGGGGCCAAAGCTTGAAG | |
| AAGGTGAATTTTCAGAAGCACGCGTAGACATCCAGATGACCCAGTCTCCATCCTCACTC | |
| TCTGCATCTACAGGAGACAGA | |
| GTCACCATCACTTGTCGGGCGAGTCAAGATATTAGCAGTTATTTAGCCTGGTATCAACAG | |
| GCACCCGGGAAAGCCCCTCA | |
| TCTCCTGATGTCTGGAGCAACCACTTTACAGACTGGAGTCCCATCAAGGTTCAGCGGCA | |
| GTGGATCTGGGACAGATTTCA | |
| CTCTCACCATCAGCAGCCTGCAGGCTGAAGATGTGGCAGTTTATTACTGTCAGCAATATT | |
| ATCGTACTCCGTTTACTTTT | |
| GGCCAGGGGACCAAGTTGGAGATCAAACGAACTGTGGCTGCACCATCTGTCTTCGCGGC | |
| CGC | |

VH18 YOL III43 Protein sequence of the total antibody protein:

| | |
|---|---|
| QVQLVQSGAELKKPGSSMKVSCKASGDTFSTYSINWVRQAPGQGLEWMGVINPSGGSTSY | (SEQ ID NO:16) |
| AQKFQGRVTMTRDTSTSTVY | |
| MELSSLRSEDTAVYYCARRRIAYGYDEGHAMDYWGQGTLVTVSSASTKGPKLEEGEFSEA | |
| RYDIQMTQSPSSLSASTGDR | |
| VTITCRASQDLSSYLAWYQQAPGKAPHLLMSGATTLQTGVPSRFSGSGSGTDFTLTISSLQAE | |
| DVAVYYCQQYYRTPFTF | |
| GQGTKLEIKRTVAAPSVFAA | |

Nucleotide sequence corresponding to VH18 YOL III43:

| | |
|---|---|
| CAGGTGCAGCTGGTGCAGTCTGGGGCTGAGTTGAAGAAGCCTGGGTCCTCGATGAAGGT | (SEQ ID NO:21) |
| CTCCTGCAAGGCTTCTGGAGA | |
| CACCTTCAGCACCTATTCTATCAACTGGGTGCGACAGGCCCCTGGACAAGGGCTTGAGT | |
| GGATGGGAGTAATCAACCCTA | |
| GTGGTGGTAGCACAAGCTACGCACAGAAGTTCCAGGGCAGAGTCACCATGACCAGGGA | |
| CACGTCCACGAGCACAGTTTAC | |
| ATGGAGCTGAGCAGCCTGAGATCTGAAGACACGGCCGTGTATTACTGTGCGAGAAGAA | |
| GAATCGCGTACGGTTACGACGA | |
| GGGCCATGCTATGGACTACTGGGGTCAAGGAACCCTTGTCACCGTCTCCTCAGCCTCCAC | |
| CAAGGGCCCAAAGCTTGAAG | |
| AAGGTGAATTTTCAGAAGCACGCGTAGACATCCAGATGACCCAGTCTCCATCCTCACTC | |
| TCTGCATCTACAGGAGACAGA | |
| GTCACCATCACTTGTCGGGCGAGTCAAGATATTAGCAGTTATTTAGCCTGGTATCAACAG | |
| GCACCCGGGAAAGCCCCTCA | |
| TCTCCTGATGTCTGGAGCAACCACTTTACAGACTGGAGTCCCATCAAGGTTCAGCGGCA | |
| GTGGATCTGGGACAGATTTCA | |
| CTCTCACCATCAGCAGCCTGCAGGCTGAAGATGTGGCAGTTTATTACTGTCAGCAATATT | |
| ATCGTACTCCGTTTACTTTT | |
| GGCCAGGGGACCAAGTTGGAGATCAAACGAACTGTGGCTGCACCATCTGTCTTCGCGGC | |
| CGC | |

### EXAMPLE 4

### Materials and Methods

Construction of a V-gene Library

Total RNA was isolated from peripheral blood lymphocytes (buffy coats) of two naive donors. mRNA was prepared with an mRNA isolation kit (Qiagen, Germany). cDNA was synthesized by oligo dT-priming. For the amplification of κ and λ light chains, a primary PCR was used applying the 5′-oligonucleotides described by Marks et al. (1991) as “human Vκ and Vλ back primers” and the 3′ oligonucleotides described as constant kappa and constant lambda primers by Welschof et al. (1995). 30 cycles with annealing at 56° C. were chosen. Secondary PCRs (maximum 14 cycles) served for adding the VL 5′ cloning site Miul and the 3′ site NotI to the first amplificates. Here, the 5′ extension TAC AGG ATC CAC GCG TA (SEQ ID NO:25) served for adding the 5′ cloning site MluI to the back primers and the 5′ extension TGA CAA GCT TGC GGC CGC (SEQ ID NO:26) added the NotI site to the constant VL primers. The resulting 2nd PCR VL amplificates were run on an agarose gel and purified with a QiaEx kit (Qiagen, Germany). To clone the VL repertoire, the phagemid vector pSEX 81 (essentially as described in Breitling et al., 1991) was overdigested with MluI and NotI. The restricted DNA was purified using QiaQuick (Qiagen, Germany) and ligated overnight with VL PCR products, overdigested with the same endonucleases. The ethanol-precipitated ligations were used to transform E. coli XL 1-Blue (Stratagene, California). Transformants were plated on 2YT plates containing 100 mM-glucose, 100 Fg/ml ampicillin, 12.5 μg/ml tetracyline and grown overnight at 30° C. Diversity of the cloned libraries was tested by BstNI-digests of PCR-amplified V regions and analysis on polyacrylamid gels. For the amplification of heavy chains, a primary PCR was used applying the 5′-oligonucleotides already described by Marks et al. (1991) as “human VH back primers” for the N-terminus of VH and the following 3′-oligonucleotides for the C-terminus of FR3 regions within the functionally rearranged gene segment families:

HU VG VH1 1/3/4: TCT CGC ACA GTA ATA CAC GGC (SEQ ID NO:27)

HU VG VH2: TCT GTG TGC ACA GTA ATA TCT GGC (SEQ ID NO:28)

HU VG VH5: TCT CGC ACA GTA ATA CAT GGC (SEQ ID NO:29)

HU VG VH6: TCT TGC ACA GTA ATA CAC AGC (SEQ ID NO:30)

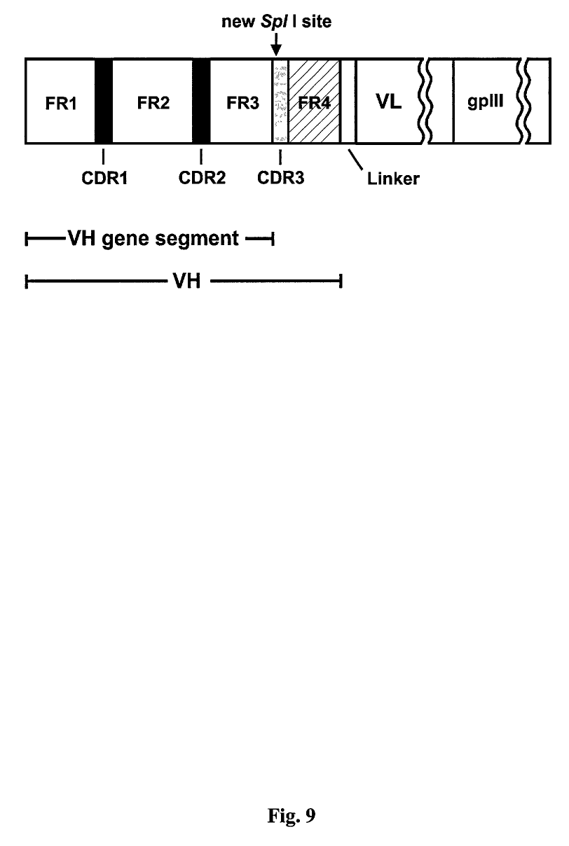

With an annealing temperature of 55-58 ° C. 30 cycles were carried out. Secondary PCRs (max . 14 cycles) served for adding the VH 5′ cloning site NcoI and the 3′ site Spll, the latter facilitating the coupling of the FR1 to FR3 gene segments with the parental HCDR3. A few microliters of the 1 st PCR were used as a template for the above primers, with the following 5′ sequences added: 5′ primers: GAA TAG GCC ATG GCG (SEQ ID NO:31). 3′ primers: GGG GGC GGG CGT ACG CGA TTC TTC T (SEQ ID NO:32). The new SplI site was inserted into the parental HCDR3 via PCR without changing the coding sense of it. This site enabled the cloning of all VH gene segment families known to be functionally rearranged (FIG. 9).

Phage Preparations and Selection

To obtain phage associated antibodies (phabs), the overinfection of exponentially growing E. coli was carried out following Schier et al. (1996). After growth at 30° C. overnight bacteria were pelleted and phages were precipitated twice with 20% polyethylene glycol in 2.5 M-NaC1. For selection 1-20 μg FAP were coated in Ma×isorb immunotubes (Nunc) rotating overnight at 4° C. After washing twice with PBS, the coated tubes were blocked with 3% non fat dry milk in PBS or with Roti-Block (Roth, Germany). Immediately before the panning, the tubes were washed twice with PBS. 10¹⁰-10¹² cfu were preadsorbed in 6% non fat dry milk (working concentration) and used for selection tumbling at RT for 2 h. In round 1 and 2 of selection, 10 to 15 washing steps with PBS followed the same number of steps with PBS-Tween 20 (0.1%). In later rounds the washing was increased to a maximum of 25 times PBS-Tween and the same number of pure PBS. For a higher stingency during washing, the Tween concentration was raised to 0.5% and considerable vortexing of the immunotubes was introduced. Elution of phages was done by either 100 mM-triethylamine or 0.1 M-HCI, pH 2.2. Eluted phages were immediately neutralized with Tris and used for infection of XL-1 Blue. After overnight growth at 30 ° C., the bacteria were scraped from the agar plates and either used for a further round of selection or frozen with glycerol.

Screening for Specific Phabs

The screening of selected phabs was carried out as described elsewhere (Mersmann et al., 1998). Briefly, we induced the expression of scFv-pIII fusion proteins without producing complete phages. These fusion proteins were tested in ELISA on purified FAP and irrelevant Ag. Binders that turned out to be FAP-specific were analyzed in competion ELISA where different amounts of a chimeric bivalent construct of the parental F19 served for synchronous competition. DNA-sequencing was done using fluorescent dideoxynucleotides and an ALFexpress (Amersham Pharmacia, Sweden) or by commercial service.

Affinity Measurements

To estimate the functional affinity of Ab constructs, their half max imal saturation concentrations were determined on FAP over-expressing fibrosarcoma cells (HT1080). 10⁵ FAP⁺ or control cells were incubated for 90 min with serial dilutions of the Ab construct. Detection was carried out by the anti-c-myc Ab 9E10 followed by an FITC labeled goat anti-mouse specific serum (in the case of scFv) or by an FITC labeled goat anti-human specific serum (in the case of minibodies (Mb)). Incubations and washings were done on ice except for the labeled Abs which were applied at RT. Bound Ab contructs were detected in a FACStar (Becton Dickinson) or in an EPICS Flow Cytometer (Coulter). The mean fluorescence was measured for 10⁴ cells in each dilution. The concentration of the applied Ab derivatives were determined in repeated estimations against a scFv or Mb standard used in SDS-PAGE and western blotting.

Cloning, Expression and Purification of Minibody (Mb)

The scFv cassettes of the selected clones 18 and 34 were excised from the scFv expression vector pOPE101 (Dutbel et al., 1992) by restriction with NcoI/NotI and inserted into an equally prepared Mb-vector, pD1, a derivative of the published vector pACK02scKan-(Pack et al., 1993). E. coli XL1-Blue were transformed as usual, subsequently, the cell wall and outer membrane deficient strain LVI of Proteus mirabilis was transformed and induced to overnight expression according to Rippmann et al. (1998). After dialysis against PBS, the Mb was ultracentrifuged (113,000×g, 4° C., 30 min) and purified by means of IMAC with a Zn²⁺ loaded HiTrap column (Pharmacia, Sweden). Fractions wered tested by SDS-PAGE and subsequent Coomassie staining.

Stability Assay for the Mb

The thermal stability of Mb #34 in RPMI medium containing 5% FCS was by incubation of purified, freshly thawed Mb at 37° C. For up to 72 h. After incubation, the solution was centrifuged (20,000×g, 4° C., 10 min) and used on immobilized FAP in an ELISA. A preceding experiment was used to determine an appropriate dilution for each of the Mb preparations to reach distinct but non-saturated ELISA signals.

Immunohistochemistry

Acetone-fixed fresh frozen sections of tumor tissues were used. The tissue section were incubated (16 h) at 4° C. with the recombinant antibodies (10 μg/ml) followed by the anti-c myc Mab 9E10 for 1 h at room temperature. Subsequently, a biotinylated horse anti-mouse serum was applied. Detection of the Ag/Ab complexes was done by the avidin-biotin immunoperoxidase method. As a negative control the section was only treated with biotinylated serum antibodies followed by the colorimetric reaction. Harris haemato×ylin was used for counterstaining of the sections.

### Results

1. Selection of Human VLs

A guided selection approach based on the scFv format was chosen for the substitution of the murine VL of the FAP specific antibody F19 first, followed by the humanization of the F19 VH. The vector pSEX81 was used, in which a VL repertoire derived from naive human donors was combined with VH F19 to obtain a combinatorial library of about 3×10⁶ different clones. This library was phage display selected on immobilized FAP to isolate human VL F19 analogues. After three rounds of selection, the screening for binders by ELISA yielded several FAP binding clones. To ensure the diversity of these isolated chimeric scFv (murine VH/human VL) their phagemid DNA was analyzed by restriction enzymes and sequenced. Various chimeric scFv (now shortly named after their VL) could be identified (III5, III10, III25, III43), consisting of the guiding VH of the paternal scFv F19 and the itemized human VLs. Table 1 shows the amino acid sequence homology of the selected light chains III5 and III43 compared to the replaced VL F19. Both listed VLs belong to the human VL subgroup kappa I according to Kabat (http:/immuno.bme.nwu.eduo), and the germline gene with the closest homology is a member of the subgroup VK-family (III5: Ve; III43: Ve). Looking at the amino acid sequence, clone III5 had as much as 64% identity in FR positions compared to the parental F19, and 59% identity in CDR positions. III43 had 69% identity in FR positions and, again, 59% identity in CDR positions compared to F19. Additionally, 115 and III43 showed a high degree of mutations compared to their putative germline genes. III5 differed in 14 amino acid positions from the sequence of the closest germline, III43 showed 17 differences (ImMunoGeneTics database: http://imgt.cnusc.fr:8104; and Cox et al., 1994).

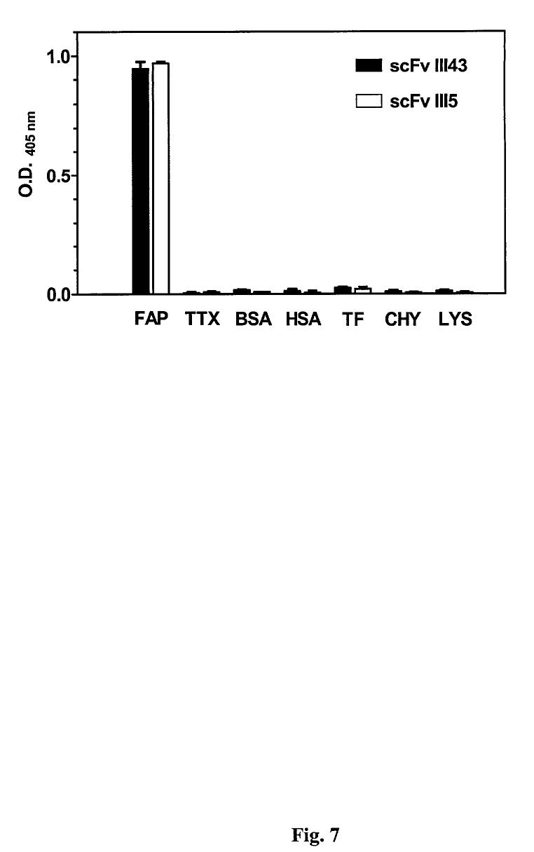

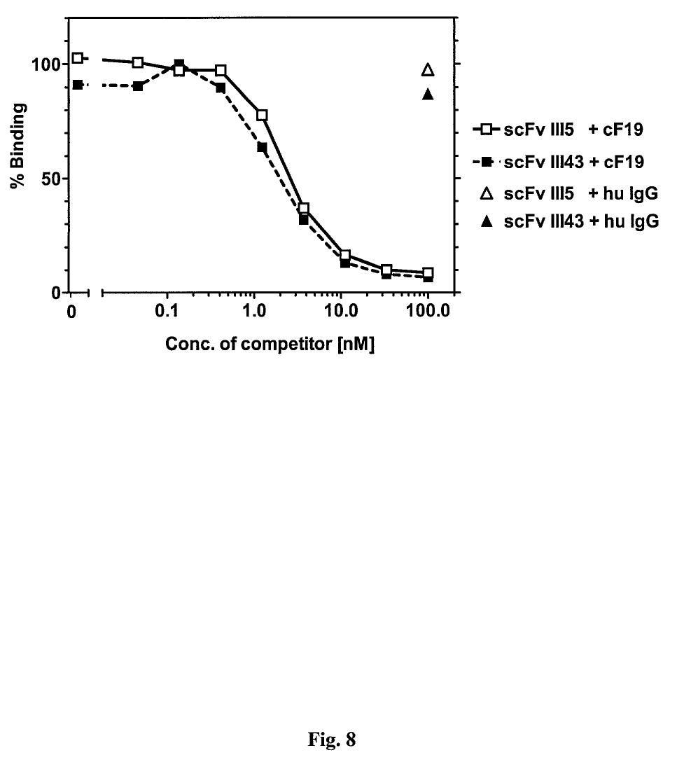

Concerning binding characteristics, the chimeric scFv were highly specific for FAP (FIG. 7). Binding competition in ELISA with cF19, a chimeric, bivalent Ab comprising the variable fragments of F19 and human constant domains, demonstrated a common epitope specificity of the selected chimeric scFvs and the parental Ab (FIG. 8). To assess the functional affinities of the selected scFv, the concentrations leading to half max imal saturation of binding (SC₅₀) were determined by sandwich ELISA using the c-myc tag for detection (Table 2). Using this assay, the parental scFv F19 had a functional affinity of 20 nM, scFv III5 of 45 nM, and scFv III43 of 20 nM. This indicates that the performed guided selection of VLs resulted in chimeric scFv of retained epitope specificity and with functional affinities in the nanomolar range.

2. Selection of Humanized VHs

In order to avoid an epitope shift during humanization of VH by guided selection as previously reported (Watzka et al., 1998), the parental HCDR3 of the murine mAb F19 was retained for subsequent selections. For this approach a phagemid vector was constructed containing HCDR3 F19, a human FR4 (found in Kabat subgroups I, II and III), and a new restriction site, which was introduced in HCDR3 without changing the amino acid sequence (FIG. 9). In this vector, the selected VL III5 and VL III43 were inserted, respectively, to encode the specific guiding structures. In a subsequent step, a cDNA derived VH segment library spanning heavy chain segments from FRI to FR3, covering rearranged sequences of all known VH germline families, was integrated into the phagemid. The resulting VH segment library (size: 4×10⁷ clones) was combined with either VL III5 or VL III43 and phage display selected on immobilized FAP.

As the selection of scFvs in phage associated form was frequently associated with strong unspecific binding, thus complicating data analyses, various selection strategies were applied (data not shown). Only highly stringent washing conditions during the panning procedure led to the isolation of two highly antigen specific, FAP-binding clones after five successive rounds of selection. In table 3, the amino acid sequences of VH clone #18 and VH clone #34 are compared with the parental VH F19 and VH OS4 (a CDR grafted version of F19). Confining the comparison to the gene segment region from FR1 to 3, the selected clone #18 showed 66% identity with the amino acid sequence of scFv F19 in the FRs, and 50% identity in the CDRs 1 and 2. For the selected clone #34 the FR identity was 67%, and 55% in CDR 1 plus 2. Both isolated VH chains use VL III43 as complement and belong to the human VH subgroup I, according to Kabat. For both VH, the closest germline gene segments were shown to belong to the VH1 segment family, which represents about 12% of all human VH gene segments (Guigou et al., 1990; Brezinschek et al., 1995). Compared to the VH 1 family (#18: DP-7, #34: DP-88), VH #18 and #34 showed 10 and 9 amino acid differences, respectively.

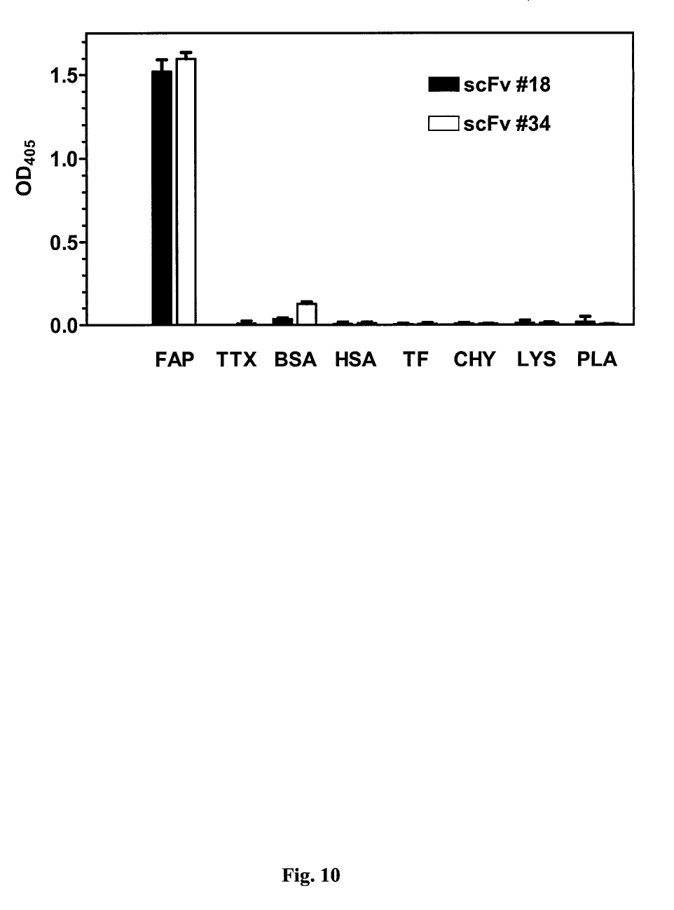

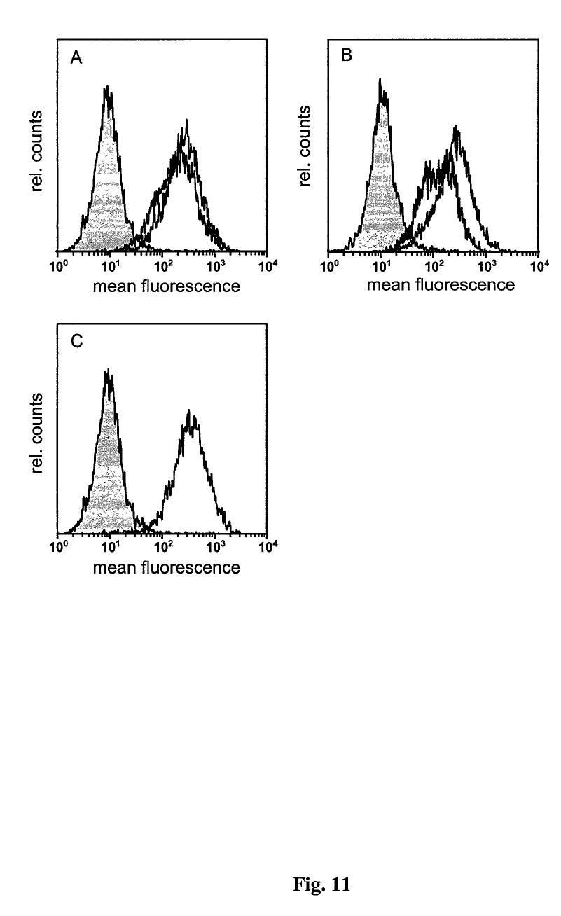

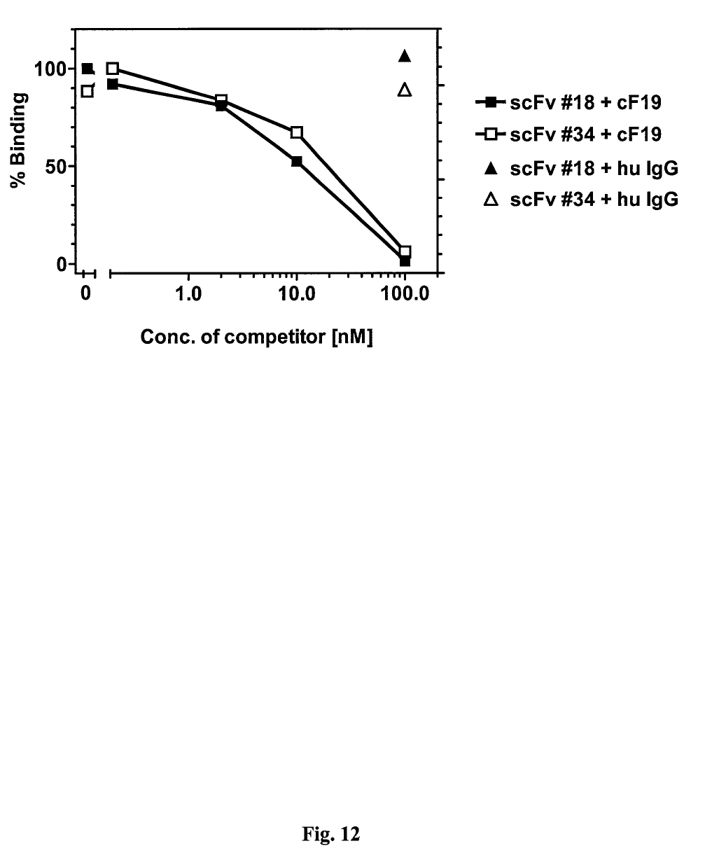

FIG. 10 shows the strict FAP-specificity of the humanized scFv #18 and #34 in ELISA. But in view of a potential clinical application of the selected human scFv, their binding characteristics to natural cell membrane expressed FAP is of particular importance. By flow cytometry we could demonstrate that scFv #18 and #34 bound to a FAP expressing human fibrosarcoma cell line, HT1080, in the same manner as the parental scFv F19 (FIG. 11). Saturation studies yielded in a functional cell binding afffinity (SC₅₀) of 6 nM for scFv #18 and scFv #34, each. In a parallel assay the SC⁵⁰ for the parental scFv F19 and its CDR grafted derivative, scFv OS4, respectively, were found to be 20 nM and 4.6 nM, indicating an even higher affinity of the selected scFv compared to the original Ab (Table 2). Moreover, binding competition of scFv #18 and #34 with cF19 was dose dependent in ELISA (data not shown) and on FAP overexpressing cells as measured by flow cytometry, demonstrating the retained epitope specificity of the selected scFvs (FIG. 12).

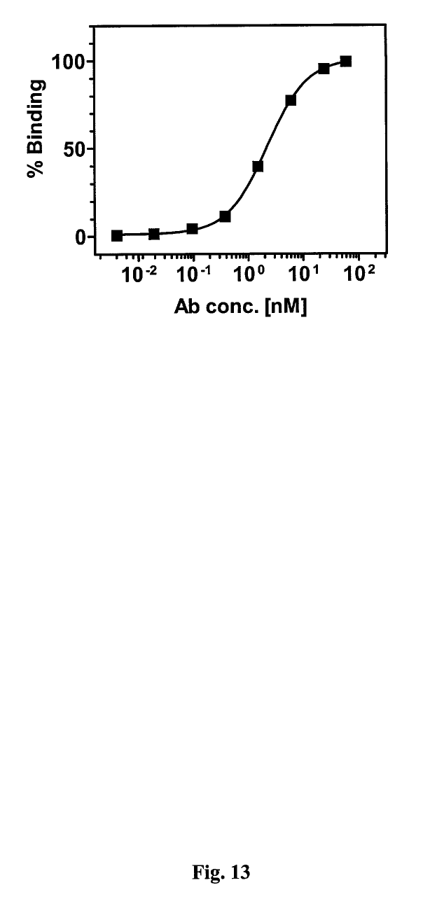

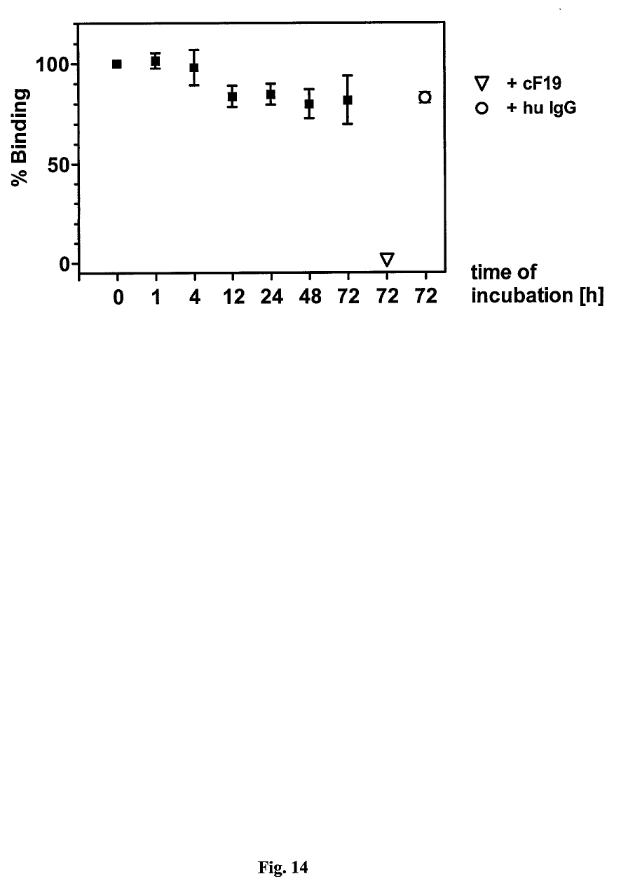

In view of potential clinical applications, the selected scFv were expressed as minibodies (Mb) using the L-form strain LVI of Proteus mirabilis (Gumpert and Taubeneck, 1983). This Ab format is advantageous for tumor targeting because of its bivalency, high tumor uptake and rapid blood clearance, resulting in a selective accumulation in the tumor (Hu et al., 1996). As expected, Mb #18 and Mb #34 exerted a high antigen specificity and retained F19 epitope specificity as demonstrated in antigen binding assays and by competition with cF19 (data not shown). Moreover, after affinity and size exclusion chromatography the functional affinity of Mb #34 on FAP-overexpressing cells was determined to be 2 nM (FIG. 13), exactly equaling the affinity assessed for the minibody version of the CDR grafted scFv OS4 (Mb OS4). Moreover, the Mb #34 turned out to have a high stability at 37° C. in serum containing media; after 72 h of incubation the loss of binding activity was only 20% (FIG. 14).

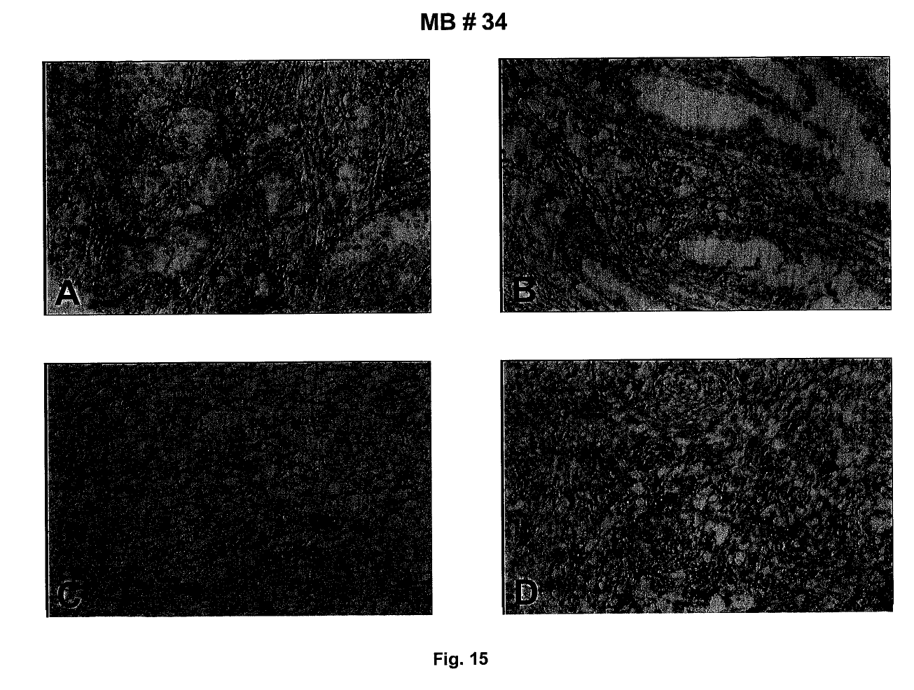

Immunohistological analyses with Mb #34 on cryo-sections of different human tumors led to a specific staining of the tumor stroma in breast, lung and colon carcinoma. Furthermore, the malignant cells of a desmoid tumor and a malignant fibrous histiocytoma could be specifically detected by Mb #34 (FIG. 15). Hence, for both, tumors of epithelial and tumors of mesenchymal origin, this human Mb exhibited an immunohistological staining pattern undistinguishable from that of F19 and Mb OS4.

The present invention is not to be limited in scope by the exemplified embodiments which are intended as illustrations of single aspects of the invention. Indeed various modifications of the invention in addition to those shown and described herein will become apparent to those skilled in the art from the foregoing description and accompanying drawings. Such modifications are intended to fall within the scope of the appended claims.

All publications and patent applications cited herein are incorporated by reference in their entireties.

what is claimed is:

1. A humanised antibody protein, which specifically binds to fibroblast activating protein alpha (FAPα) wherein the antibody protein is fully human.

2. A humanised antibody protein, which specifically binds to fibroblast activating protein alpha (FAPα), comprising not more than one murine complementarity-determining region (CDR region) of the monoclonal antibody F19 (ATCC accession number HB 8269).

3. The antibody protein according to claim 1 or 2, comprising a heavy chain (V_{H}) of the class IgM.

4. The antibody protein according to claim 1 or 2, comprising a heavy chain (V_{H}) of the class IgG.

5. The antibody protein according to claim 1 or 2, comprising a heavy chain (V_{H}) of the class IgD.

6. The antibody protein according to claim 1 or 2, comprising a light chain (V_{L}) of the lambda type λ

7. The antibody protein according to claim 1 or 2, comprising a light chain (V_{L}) of the kappa type (κ).

8. The antibody protein according to claim 1 or 2, wherein it is a Fab fragment.

9. The antibody protein according to claim 1 or 2, wherein it is an F(ab′)2 fragment.

10. The antibody protein according to claim 1 or 2, wherein it is a single-chain-Fv protein (scFv).

11. The antibody protein according to claim 1 or 2, wherein it is a diabody antibody fragment.

12. The antibody protein according to claim 1 or 2, wherein it is a minibody antibody fragment.

13. The antibody protein according to claim 1 or 2, wherein it is a multimerised antibody fragment.

14. The antibody protein according to claim 2, wherein it is fully human.

15. The antibody protein according to claim 1 or 2, wherein the variable region of the heavy chain (V_{H}) comprises the amino acid sequence according to SEQ ID NO: 1 (VH13).

16. The antibody protein according to claim 1 or 2, wherein the variable region of the heavy chain (V_{H}) comprises the amino acid sequence according to SEQ ID NO:2 (VH46).

17. The antibody protein according to claim 1 or 2, wherein the variable region of the heavy chain (V_{H}) comprises the amino acid sequence according to SEQ ID NO:3 (VH50).

18. The antibody protein according to claim 1 or 2, wherein the variable region of the light chain (V_{L}) comprises the amino acid sequence according to SEQ ID NO:4 (VLIII25).

19. The antibody protein according to claim 1 or 2, wherein the variable region of the heavy chain (V_{H}) is coded by the nucleotide seq-uence according to SEQ ID NO: 5 (VH13) or by fragments or degenerate variants thereof.

20. The antibody protein according to claim 1 or 2, wherein the variable region of the heavy chain (V_{H}) is coded by the nucleotide sequence according to SEQ ID NO:6 (VH46) or by fragments or degenerate variants thereof.

21. The antibody protein according to claim 1 or 2, wherein the variable region of the heavy chain (V_{H}) is coded by the nucleotide sequence according to SEQ ID NO:7 (VH50) or by fragments or degenerate variants thereof.

22. The antibody protein according to claim 1 or 2, wherein the variable region of the light chain (V_{L}) is coded by the nucleotide sequence according to SEQ ID NO:8 (VLIII25) or by fragments or degenerate variants thereof.

23. An antibody protein, wherein the variable region of the heavy chain (V_{H}) comprises the amino acid sequence according to SEQ ID NO: 1 (VH13) and the variable region of the light chain (V_{H}) comprises the amino acid sequence according to SEQ ID NO:4 (VLIII25).

24. An antibody protein, wherein the coding sequence of the variable region of the heavy chain (V_{H}) comprises the nucleotide sequence according to SEQ ID NO:5 (VH13) and the coding sequence of the variable region of the light chain (V_{L}) comprises the nucleotide sequence according to SEQ ID NO:8 (VLIII25).

25. An antibody protein, wherein the variable region of the heavy chain (V_{H}) comprises the amino acid sequence according to SEQ ID NO:2 (VH46) and the variable region of the light chain (V_{L}) comprises the amino acid sequence according to SEQ ID NO:4 (VLIII25).

26. An antibody protein, wherein the coding sequence of the variable region of the heavy chain (V_{H}) comprises the nucleotide sequence according to SEQ ID NO:6 (VH46) and the coding sequence of the variable region of the light chain (V_{L}) comprises the nucleotide sequence according to SEQ ID NO:8 (VLIII25).

27. An antibody protein, wherein the variable region of the heavy chain (V_{H}) comprises the amino acid sequence according to SEQ ID NO:3 (VH50) and the variable region of the light chain (V_{L}) comprises the amino acid sequence according to SEQ ID NO:4 (VLIII25).

28. An antibody protein, wherein the coding sequence of the variable region of the heavy chain (V_{H}) comprises the nucleotide sequence according to SEQ ID NO:7 (VH50) and the coding sequence of the variable region of the light chain (V_{L}) comprises the nucleotide sequence according to SEQ ID NO:8 (VLIII25).

29. The antibody protein according to claim 2, wherein the CDR comprises murine CDR 1 of the light chain (V_{L}) of the monoclonal antibody F19.

30. The antibody protein according to claim 2, wherein the CDR comprises murine CDR 2 of the light chain (V_{L}) of the monoclonal antibody F19.

31. The antibody protein according to claim 2, wherein the CDR comprises murine CDR 3 of the light chain (V_{L}) of the monoclonal antibody F19.

32. The antibody protein according to claim 2, wherein the CDR comprises murine CDR 1 of the heavy chain (V_{H}) of the monoclonal antibody F19.

33. The antibody protein according to claim 2, wherein the CDR comprises murine CDR 2 of the heavy chain (V_{H}) of the monoclonal antibody F19.

34. The antibody protein according to claim 2, wherein the CDR comprises murine CDR 3 of the heavy chain (V_{H}) of the monoclonal antibody F19.

35. The antibody protein according to claim 1 or 2, wherein the variable region of the heavy chain (V_{H}) comprises the amino acid sequence according to SEQ ID NO:9 (VH34).

36. The antibody protein according to claim 1 or 2, wherein the variable region of the heavy chain (V_{H}) comprises the amino acid sequence according to SEQ ID NO: 10 (VH18).

37. The antibody protein according to claim 1 or 2, wherein the variable region of the light chain (V_{L}) comprises the amino acid sequence according to SEQ ID NO: 11 (VLIII43).

38. The antibody protein according to claim 1 or 2, wherein the variable region of the heavy chain (V_{H}) is coded by the nucleotide sequence according to SEQ NO: 12 (VH34) or by fragments or degenerate variants thereof.

39. The antibody protein according to claim 1 or 2, wherein the variable region of the heavy chain (V_{H}) is coded by the nucleotide sequence according to SEQ NO: 13 (VH18) or by fragments or degenerate variants thereof.

40. The antibody protein according to claim 1 or 2, wherein the variable region of the light chain (V_{L}) is coded by the nucleotide sequence according to SEQ NO: 14 (VLIII43) or by fragments or degenerate variants thereof.

41. An antibody protein, wherein the variable region of the heavy chain (V_{H}) comprises the acid lo sequence according to SEQ ID NO:9 (VH34) and the variable region of the light chain (V_{H}) comprises the amino acid sequence according to SEQ ID NO: 11 (VLIII43).

42. An antibody protein, wherein the variable region of the heavy chain (V_{H}) comprises the nucleotide sequence according to SEQ ID NO: 12 (VH34) and the coding sequence of the variable region of the light chain (V_{L}) comprises the nucleotide sequence according to SEQ ID NO: 14 (VLIII43).

43. An antibody protein, wherein the variable region of the heavy chain (V_{H}) comprises the amino acid sequence ID NO: 10 (VH18) and the variable region of the light chain V_{L}) comprises the amino acid sequence ID NO: 11 (VLIII43).

44. An antibody protein, wherein the coding sequence of the variable region of the heavy chain (V_{H}) comprises the nucleotide sequence ID NO: 13 (VH 18) and the coding sequence of the variable region of the light chain (V_{L}) comprises the nucleotide sequence ID NO: 14 (VLIII43).

45. A nucleic acid comprising a nucleotide sequence encoding an antibody protein according to claim 1 or 2.

46. A recombinant DNA vector comprising a nucleic acid according to claim 45.

47. The recombinant DNA vector according to claim 46, which is an expression vector.

48. A host cell comprising a vector according to claim 46.

49. The host cell according to claim 48, which is a eukaryotic host cell.

50. The host cell according to claim 48 or 49, which is a mammalian cell.

51. The host cell according to claim 50, which is a BHK, CHO or COS cell.

52. The host cell according to claim 48, which is a bacteriophage.

53. The host cell according to claim 48, which is a prokaryotic host cell.

54. A process for preparing a humanized antibody protein which specifically binds to fibroblast activating protein alpha (FAPα), comprising: cultivating a host cell according to one of claims 48 to 51 under conditions in which said antibody protein is expressed by said host cell and isolating said antibody protein.

55. The process according to claim 54, wherein said host is a mammalian cell.

56. The process according to claim 55, wherein said host is a CHO or COS cell.

57. The process according to claim 54, wherein said host cell is co-transfected with two plasmids which carry the expression units for the light or the heavy chain.

58. The antibody protein according to claim 1 or 2, wherein said antibody protein is coupled to a therapeutic agent.

59. The antibody protein according to claim 58, wherein said therapeutic agent is selected from among the radioisotopes, toxins, toxoids, boron, fusion proteins, inflammatory agents and chemotherapeutic agents.

60. The antibody protein according to claim 59, wherein said radioisotope is a β-emitting radioisotope.

61. The antibody protein according to claim 60, wherein said radioisotope is selected from among ¹⁸⁶rhenium, ¹⁸⁸rhenium, ¹³¹iodine and ⁹⁰yttrium.

62. The antibody protein according to claim 1 or 2, wherein said antibody protein is labelled.

63. The antibody protein according to claim 62, which is labelled with a detectable marker.

64. The antibody protein according to claim 63, wherein the detectable marker is selected from among the enzymes, dyes, radioisotopes, digoxygenine, streptavidine and biotin.

65. The antibody protein according to claim 1 or 2, wherein the antibody protein is coupled to an imageable agent.

66. The antibody protein according to claim 65, wherein the imageable agent is a radioisotope.

67. The antibody according to claim 66, wherein said radioisotope is a β-emitting radioisotope.

68. The antibody protein according to claim 67, wherein said radioisotope is ¹²⁵iodine.

69. A pharmaceutical composition, comprising an antibody protein according to claim 1 or 2; and a pharmaceutically acceptable carrier.

70. A pharmaceutical preparation, comprising an antibody protein according to claim 58; and a pharmaceutically acceptable carrier.

71. A pharmaceutical preparation, comprising an antibody protein according to claim 65; and a pharmaceutically acceptable carrier.

72. A method for the treatment or imaging of a tumor comprising contacting a tumor with the preparation according to claim 69 wherein said tumour is associated with activated stromal fibroblasts.

73. A method for the treatment or imaging of a tumour comprising contacting a tumor with the preparation according to claim 70 wherein said tumor is associated with activated stromal fibroblasts.

74. A method for the treatment or imaging of a tumor comprising contacting a tumor with the preparation according to claim 71 wherein said tumor is associated with activated stromal fibroblasts.

75. The method according to claim 72 wherein said tumours are selected from among colorectal cancer, non-small-cell lung cancer, breast cancer, head and neck cancer, ovarian cancer, lung cancer, bladder cancer, pancreatic cancer and metastatic brain cancer.

76. A process for detecting activated stromal fibroblasts in wound healing, inflammatory processes or in a tumor, comprising contacting a probe, which might possibly contain activated fibroblasts with an antibody protein according to claim 1 or 2 under conditions which are suitable for forming a complex from said antibody protein with its antigen and detecting the formation of said complex.

77. A process for detecting activated stromal fibroblasts in wound healing, inflammatory processes or in a tumor, comprising contacting a probe, which might possibly contain activated fibroblasts with an antibody protein according to claim 62 under conditions which are suitable for forming a complex from said antibody protein with its antigen and detecting the formation of said complex.

78. The process according to claim 77, wherein said tumour is selected from among colorectal cancer, non-small-cell lung cancer, breast cancer, head and neck cancer, ovarian cancer, lung cancer, bladder cancer, pancreatic cancer and metastatic brain cancer.

79. A process for detecting tumour stroma, comprising contacting a suitable probe is with an antibody protein according to claim 1 or 2 under suitable conditions for the formation of an antibody-antigen comple×; detecting the complex thus formed; and correlating the presence of the complex thus formed with the presence of tumour stroma.

80. A process for detecting tumour stroma, comprising contacting a suitable probe is with an antibody protein according to claim 62 under suitable conditions for the formation of an antibody-antigen complex ; detecting the comple×thus formed; and correlating the presence of the complex thus formed with the presence of tumour stroma.

81. An antibody protein comprising an amino acid sequence according to sequence ID NO: 15 or a part thereof or a functional variant thereof.

82. An antibody protein comprising an amino acid sequence according to sequence ID NO: 16 or a part thereof or a functional variant thereof.

83. An antibody protein comprising an amino acid sequence according to sequence ID NO:17 or a part thereof or a functional variant thereof.

84. An antibody protein comprising an amino acid sequence according to sequence ID NO: 18 or a part thereof or a functional variant thereof.

85. An antibody protein comprising an amino acid sequence according to sequence ID NO: 19 or a part thereof or a functional variant thereof.

86. An antibody protein which is coded by a nucleotide sequence according to sequence ID NO:20 or a part thereof or a functional variant thereof.

87. An antibody protein which is coded by a nucleotide sequence according to sequence ID NO:21 or a part thereof or a functional variant thereof.

88. An antibody protein which is coded by a nucleotide sequence according to sequence ID NO:22 or a part thereof or a functional variant thereof.

89. An antibody protein which is coded by a nucleotide sequence according to sequence ID NO:23 or a part thereof or a functional variant thereof.

90. An antibody protein which is coded by a nucleotide sequence according to sequence ID NO:24 or a part thereof or a functional variant thereof.

91. An antibody protein consisting of the amino acid sequence according to SEQ ID NO:15.

92. An antibody protein consisting of the amino acid sequence according to SEQ ID NO:16.

93. An antibody protein consisting of the amino acid sequence according to SEQ ID NO:17.

94. An antibody protein consisting of the amino acid sequence according to SEQ ID NO:18.

95. An antibody protein consisting of the amino acid sequence according to SEQ ID NO:19.

96. An antibody protein consisting of the amino acid sequence according to SEQ ID NO:20.

97. An antibody protein which is coded by the nucleotide sequence according to SEQ ID NO:21.

98. An antibody protein which is coded by the nucleotide sequence according to SEQ ID NO:22.

99. An antibody protein which is coded by the nucleotide sequence according to SEQ ID NO:23.

100. An antibody protein which is coded by the nucleotide sequence according to SEQ ID NO:24.
